(19) United States
(12) Patent Application Publication
(10) Pub. No.: US 2015/0265723 A1
(43) Pub. Date: Sep. 24, 2015

(54) METHODS OF TREATING METASTATIC BREAST CANCER WITH TRASTUZUMAB-MCC-DM1

(71) Applicant: Genentech, Inc., South San Francisco, CA (US)

(72) Inventors: Samuel Agresta, South San Francisco, CA (US); Barbara Klencke, South San Francisco, CA (US)

(21) Appl. No.: 14/727,728

(22) Filed: Jun. 1, 2015

Related U.S. Application Data
(63) Continuation of application No. 12/959,975, filed on Dec. 3, 2010, now abandoned.
(60) Provisional application No. 61/266,848, filed on Dec. 4, 2009.

(51) Int. Cl. A61K 47/48; A61K 47/26; A61K 47/12; A61K 31/537

(52) U.S. Cl. CPC A61K 47/48561; A61K 31/537; A61K 47/48584; A61K 47/26; A61K 47/12

(57) ABSTRACT

Methods of treating patients having metastatic or unresectable locally advanced HER2 positive cancer, e.g., breast cancer, with the antibody-drug conjugate trastuzumab-MCC-DM1 are provided, wherein the patients have received extensive prior treatment, e.g., with an anthracycline, a taxane, capecitabine, lapatinib, and trastuzumab.

### RELATED APPLICATIONS

This application is a continuation of U.S. application Ser. No. 12/959,975, filed Dec. 3, 2010, which claims the benefit of U.S. Provisional Application No. 61/266,848, filed on Dec. 4, 2009, the entire contents of which are incorporated herein by reference.

### FIELD OF THE INVENTION

The invention relates to methods of using Trastuzumab-MCC-DM1 for the treatment of metastatic or unresectable locally advanced breast cancer.

### BACKGROUND OF THE INVENTION

The HER2 (ErbB2) receptor tyrosine kinase is a member of the epidermal growth factor receptor (EGFR) family of transmembrane receptors. Overexpression of HER2 is observed in approximately 20% of human breast cancers (hereinafter referred to as HER2-positive breast cancer) and is implicated in the aggressive growth and poor clinical outcomes associated with these tumors (Slamon et al (1987) Science 235:177-182). HER2 protein overexpression can be determined using an immunohistochemistry based assessment of fixed tumor blocks (Press M F, et al (1993) Cancer Res 53:4960-70).

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. No. 5,677,171; U.S. Pat. No. 5,821,337; U.S. Pat. No. 6,054,297; U.S. Pat. No. 6,165,464; U.S. Pat. No. 6,339,142; U.S. Pat. No. 6,407,213; U.S. Pat. No. 6,639,055; U.S. Pat. No. 6,719,971; U.S. Pat. No. 6,800,738; U.S. Pat. No. 7,074,404; Coussens et al (1985) Science 230:1132-9; Slamon et al (1989) Science 244:707-12; Slamon et al (2001) New Engl. J. Med. 344:783-792). Trastuzumab has been shown, in both in vitro assays and in animals, to inhibit the proliferation of human tumor cells that overexpress HER2 (Hudziak et al (1989) Mol Cell Biol 9:1165-72; Lewis et al (1993) Cancer Immunol Immunother; 37:255-63; Baselga et al (1998) Cancer Res. 58:2825-2831). Trastuzumab is a mediator of antibody-dependent cellular cytotoxicity, ADCC (Lewis et al (1993) Cancer Immunol Immunother 37(4):255-263; Hotaling et al (1996) [abstract]. Proc. Annual Meeting Am Assoc Cancer Res; 37:471; Pegram M D, et al (1997) [abstract]. Proc Am Assoc Cancer Res; 38:602; Sliwkowski et al (1999) Seminars in Oncology 26(4), Suppl 12:60-70; Yarden Y. and Sliwkowski, M. (2001) Nature Reviews: Molecular Cell Biology, Macmillan Magazines, Ltd., Vol. 2:127-137).

HERCEPTIN® was approved in 1998 for the treatment of patients with ErbB2-overexpressing metastatic breast cancers (Baselga et al, (1996) J. Clin. Oncol. 14:737-744) that have received extensive prior anti-cancer therapy, and has since been used in over 300,000 patients (Slamon D J, et al. N Engl J Med 2001; 344:783-92; Vogel C L, et al. J Clin Oncol 2002; 20:719-26; Marty M, et al. J Clin Oncol 2005; 23:4265-74; Romond E H, et al. T N Engl J Med 2005; 353:1673-84; Piccart-Gebhart M J, et al. N Engl J Med 2005; 353:1659-72; Slamon D, et al. [abstract]. Breast Cancer Res Treat 2006, 100 (Suppl 1): 52). In 2006, the FDA approved HERCEPTIN® (trastuzumab, Genentech Inc.) as part of a treatment regimen containing doxorubicin, cyclophosphamide and paclitaxel for the adjuvant treatment of patients with HER2-positive, node-positive breast cancer. While the development of HERCEPTIN® provided patients with HER2-positive tumors a markedly better outcome than with chemotherapy alone, virtually all HER2-positive, metastatic breast cancer (MBC) patients will eventually progress on available therapies. Opportunities remain to improve outcomes for patients with MBC. Despite trastuzumab's diverse mechanisms of action, a number of patients treated with trastuzumab show either no response or stop responding after a period of treatment benefit. Some HER2 positive tumors fail to respond to HERCEPTIN® and the majority of patients whose tumors respond eventually progress. There is a significant clinical need for developing further HER2-directed cancer therapies for patients with HER2-overexpressing tumors or other diseases associated with HER2 expression that do not respond, or respond poorly, to HERCEPTIN® treatment.

An alternative approach to antibody-targeted therapy is to utilize antibodies for delivery of cytotoxic drugs specifically to antigen-expressing cancer cells. Antibody-drug conjugates, or ADCs, are monoclonal antibodies to which highly potent cytotoxic agents have been conjugated. ADCs represent a novel approach to conferring tumor selectivity on systemically administered anti-tumor therapeutics. Utilizing surface antigens that are tumor-specific and/or overexpressed, ADCs are designed to focus the delivery of highly potent cytotoxic agents to tumor cells. The potential of this approach is to create a more favorable therapeutic window for such agents than could be achieved by their administration as free drugs.

Maytansinoids, derivatives of the anti-mitotic drug maytansine, bind to microtubules in a manner similar to vinca alkaloid drugs (Issell B F et al (1978) Cancer Treat. Rev. 5:199-207; Cabanillas F et al. (1979) Cancer Treat Rep, 63:507-9. ADCs composed of the maytansinoid DM1 linked to trastuzumab show potent anti-tumor activity in HER2-overexpressing trastuzumab-sensitive and trastuzumab-resistant tumor cell lines, and xenograft models of human breast cancer. A conjugate of maytansinoids linked to the anti-HER2 murine breast cancer antibody TA.1 via the MCC linker was 200-fold less potent than the corresponding conjugate with a disulfide linker (Chari et al (1992) Cancer Res. 127-133).

Trastuzumab-MCC-DM1 (trastuzumab-DM1; T-DM1; PRO132365), a novel antibody-drug conjugate (ADC) specifically designed for the treatment of HER2-positive breast cancer, is composed of the cytotoxic agent DM1 (a thiol-containing maytansinoid anti-microtubule agent) conjugated to trastuzumab via lysine side chains, with an average drug to antibody ratio of about 3.4:1. T-DM1 is in development for the treatment of HER2+ metastatic breast cancer (Beeram M, Burris H, Modi S et al. (2008) J Clin Oncol 26: May 20 suppl; abstr 1028). T-DM1 binds to HER2 with affinity similar to that of trastuzumab, and such binding is required for T-DM1 anti-tumor activity (Trastuzumab (Herceptin2.®) Investigator Brochure, Genentech, Inc., South San Francisco, Calif., July 2007). It is hypothesized that after binding to HER2, T-DM1 undergoes receptor-mediated internalization, resulting in intracellular release of DM1 and subsequent cell death (Austin C D, De Mazière A M, Pisacane P I, et al. (2004) Mol Biol Cell 15(12):5268-5282).

DM1 is a thiol-containing maytansinoid derived from the naturally occurring ester ansamitocin P3 (Remillard S, Rebhun L I, Howie G A, et al. (1975) Science 189(4207):1002-1005.3; Cassady J M, Chan K K, Floss H G. (2004) Chem Pharm Bull 52(1):1-26.4). The related plant ester, maytansine, has been studied as a chemotherapeutic agent in approximately 800 patients, administered at a dose of 2.0 mg/m² every 3 weeks either as a single dose or for 3 consecutive days (Issell B F, Crooke S T. (1978) Maytansine. Cancer Treat Rev 5:199-207). Despite nonclinical activity, the activity of maytansine in the clinic was modest at doses that could be safely delivered. The dose-limiting toxicity (DLT) was gastrointestinal, consisting of nausea, vomiting, and diarrhea (often followed by constipation). These toxicities were dose dependent but not schedule dependent. Peripheral neuropathy (predominantly sensory) was reported and was most apparent in patients with preexisting neuropathy. Subclinical transient elevations of hepatic transaminase, alkaline phosphatase, and total bilirubin were reported. Constitutional toxicities, including weakness, lethargy, dysphoria, and insomnia, were common. Less common toxicities included infusion-site phlebitis and mild myelosuppression. Further development of the drug was abandoned in the 1980s because of the narrow therapeutic window.

Clinical results to date suggest that T-DM1 may benefit patients with HER2-positive MBC who have progressed while receiving HER2-directed therapy. Trastuzumab-MCC-DM1 (T-DM1) is currently undergoing evaluation in phase II clinical trials in patients whose disease is refractory to HER2-directed therapies (Beeram et al (2007) “A phase I study of trastuzumab-MCC-DM1 (T-DM1), a first-in-class HER2 antibody-drug conjugate (ADC), in patients (pts) with HER2+ metastatic breast cancer (BC)”, American Society of Clinical Oncology 43rd:June 02 (Abs 1042; Krop et al, European Cancer Conference ECCO, Poster 2118, Sep. 23-27, 2007, Barcelona; U.S. Pat. No. 7,097,840; US 2005/0276812; US 2005/0166993).

In a Phase I study, single-agent T-DM1 administered by IV infusion every 3 weeks was evaluated at doses ranging from 0.3 to 4.8 mg/kg in 24 patients with HER2-positive MBC. There was intensive PK sampling in the first cycle and additional pre-dose and post-dose samples in other cycles in all patients, including the 15 patients treated at the MTD (3.6 mg/kg, the dose to be used in this trial). In an ongoing Phase II study, PK data are being obtained from all patients treated at 3.6 mg/kg.

In this Phase I Study, the maximum tolerated dose (MTD) of T-DM1 administered by IV infusion every 3 weeks was 3.6 mg/kg. A DLT (Dose-Limiting Toxicity) consisted of Grade 4 thrombocytopenia in 2 of 3 patients treated at 4.8 mg/kg. Related Grade ≧□2 adverse events at 3.6 mg/kg were infrequent and manageable. This treatment schedule was well tolerated and associated with significant clinical activity as described previously (Beeram et al (2007) “A phase I study of trastuzumab-MCC-DM1 (T-DM1), a first-in-class HER2 antibody-drug conjugate (ADC), in patients (pts) with HER2+ metastatic breast cancer (BC)”, American Society of Clinical Oncology 43rd:June 02 (Abs 1042; Krop et al, European Cancer Conference ECCO, Poster 2118, Sep. 23-27, 2007, Barcelona). A Phase II study has shown similar tolerability at the 3.6 mg/kg dose level administered every 3 weeks, with only a small percentage of patients (3 out of 112 patients) requiring dose reduction (Vogel, C. L. et al “A phase II study of trastuzumab-DM1 (T-DM1), a HER2 antibody-drug conjugate (ADC), in patients (pts) with HER2+ metastatic breast cancer (MBC): Final results” (2009) J Clin Oncol 27:15s, 2009 (suppl; abstr 1017), 2009 ASCO).

Thus, the T-DM1 dose of 3.6 mg/kg administered every 3 weeks was selected for testing in the Phase II study described herein based on: (1) the demonstrated efficacy and safety of T-DM1 at 3.6 mg/kg every 3 weeks, and (2) the convenience of a 3-week regimen for this patient population.

### SUMMARY OF THE INVENTION

The invention relates generally to methods of treating breast cancer patients with the antibody-drug conjugate, trastuzumab-MCC-DM1 (T-DM1).

In one aspect, a method for the treatment of metastatic or unresectable locally advanced HER2 positive cancer in a patient comprising administering a therapeutically effective amount of trastuzumab-MCC-DM1 is provided, wherein the patient has been previously treated with at least two anti-HER2 agents. In one embodiment, the at least two anti-HER2 agents are lapatinib and trastuzumab. In any of the above embodiments, the patient has been previously treated with an anthracycline, a taxane, and capecitabine. In any of the above embodiments, the cancer is breast cancer. In any of the above embodiments, trastuzumab-MCC-DM1 is administered at three week intervals to the patient. In any of the above embodiments, the therapeutically effective amount of trastuzumab-MCC-DM1 is 1 mg to 10 mg/kg/day of patient body weight. In any of the above embodiments, trastuzumab-MCC-DM1 is formulated with sodium succinate, pH 5.0, and 0.02% (w/v) polysorbate 20. In any of the above embodiments, trastuzumab-MCC-DM1 is formulated with 6% (w/v) trehalose dihydrate or 6% (w/v) sucrose.

In a further aspect, a method for the treatment of metastatic or unresectable locally advanced HER2 positive cancer in a patient is provided, the method comprising administering a therapeutically effective amount of trastuzumab-MCC-DM1, wherein the patient was previously treated with two or more therapies selected from an anthracycline, a taxane, capecitabine, lapatinib, and trastuzumab, and wherein the patient progressed on their most recent treatment. In one embodiment, the cancer is breast cancer. In any of the above embodiments, trastuzumab-MCC-DM1 is administered at three week intervals to the patient. In any of the above embodiments, the therapeutically effective amount of trastuzumab-MCC-DM1 is 1 mg to 10 mg/kg/day of patient body weight. In any of the above embodiments, trastuzumab-MCC-DM1 is formulated with sodium succinate, pH 5.0, and 0.02% (w/v) polysorbate 20. In any of the above embodiments, trastuzumab-MCC-DM1 is formulated with 6% (w/v) trehalose dihydrate or 6% (w/v) sucrose.

### BRIEF DESCRIPTION OF THE DRAWINGS

FIG. 1 shows Mean (±SD) Pharmacokinetic Parameters for T-DM1 following IV Administration of T-DM1 Every Three Weeks (Cycle 1).

FIG. 2 shows a plot of Mean (±SD) Serum or Plasma T-DM1, Total Trastuzumab, and Free DM1 Concentrations in Cycle 1 following 3.6 mg/kg T-DM1 Every Three Weeks.

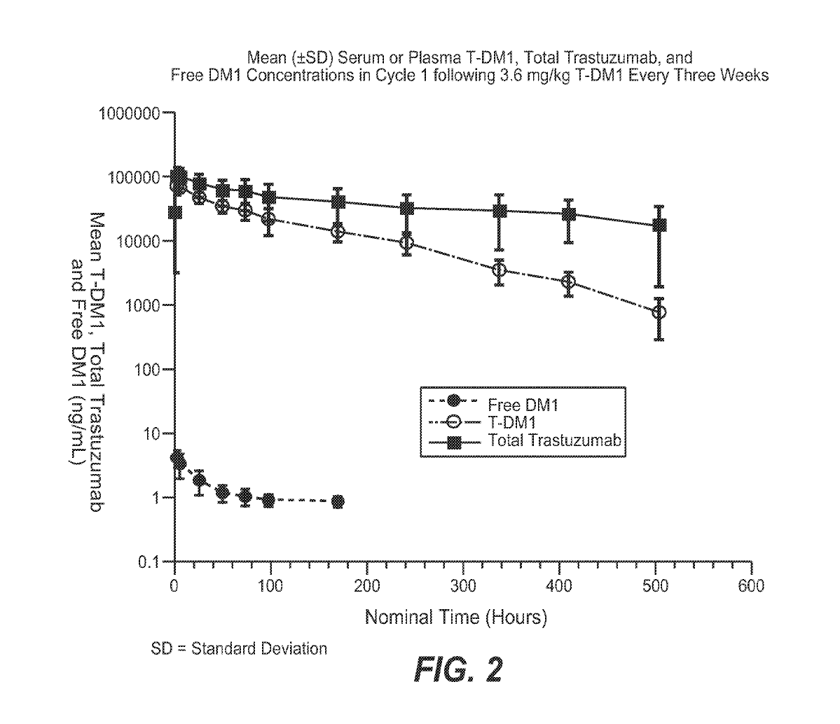

FIG. 3 shows a Kaplan-Meier plot of progression-free survival (PFS) in all treated patients.

FIG. 4 shows a Kaplan-Meier plot of progression-free survival (PFS) in patients separated by HER2 status: HER2 normal and HER2 positive (+).

FIG. 5 shows a Kaplan-Meier plot of PFS per IRF Assessment by HER2 qRT-PCR for Retrospectively Confirmed HER2+ treated patients.

### DETAILED DESCRIPTION OF EXEMPLARY EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

### DEFINITIONS

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of a hyperproliferative condition, such as cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The term “metastatic breast cancer” means the state of breast cancer where the cancer cells are transmitted from the original site to one or more sites elsewhere in the body, by the blood vessels or lymphatics, to form one or more secondary tumors in one or more organs besides the breast.

“Progression-Free Survival” (PFS) is the time from the first day of treatment to documented disease progression (including isolated CNS progression) or death from any cause on study, whichever occurs first.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy. Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine,dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Il), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gammalI, calicheamicin omegall (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKO polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside (Ara-C); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE0); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

An “adverse event” is any unfavorable and unintended sign, symptom, or disease temporally associated with the use of an investigational (medicinal) product or other protocol-imposed intervention, regardless of attribution; and includes: AEs not previously observed in the patient that emerge during the protocol-specified AE reporting period, including signs or symptoms associated with breast cancer that were not present before the AE reporting period; complications that occur as a result of protocol-mandated interventions (e.g., invasive procedures such as biopsies); if applicable, AEs that occur before assignment of study treatment associated with medication washout, no treatment run-in, or other protocol-mandated intervention; Preexisting medical conditions (other than the condition being studied) judged by the investigator to have worsened in severity or frequency or changed in character during the protocol-specified AE reporting period

An adverse event is classified as a “Serious Adverse Events” (SAE) if it meets the following criteria: results in death (i.e., the AE actually causes or leads to death); life threatening (i.e., the AE, in the view of the investigator, places the patient at immediate risk of death, but not including an AE that, had it occurred in a more severe form, might have caused death); requires or prolongs inpatient hospitalization; results in persistent or significant disability/incapacity (i.e., the AE results in substantial disruption of the patient's ability to conduct normal life functions); results in a congenital anomaly/birth defect in a neonate/infant born to a mother exposed to the investigational product; or is considered a significant medical event by the investigator based on medical judgment (e.g., may jeopardize the patient or may require medical/surgical intervention to prevent one of the outcomes listed above). All AEs that do not meet any of the criteria for serious are regarded as non-serious AEs. The terms “severe” and “serious” are not synonymous. Severity (or intensity) refers to the grade of a specific AE, e.g., mild (Grade 1), moderate (Grade 2), or severe (Grade 3) myocardial infarction (see Section 5.2.2). “Serious” is a regulatory definition (see previous definition) and is based on patient or event outcome or action criteria usually associated with events that pose a threat to a patient's life or functioning. Seriousness (not severity) serves as the guide for defining regulatory reporting obligations from the Sponsor to applicable regulatory authorities. Severity and seriousness should be independently assessed when recording AEs and SAES on the eCRF

### List of Abbreviations and Definition of Terms

| Abbreviation | Definition |
|---|---|
| ADC | antibody-drug conjugate |
| AE | adverse event |
| CBR | clinical benefit rate |
| CHF | congestive heart failure |
| CL | clearance |
| CNS | central nervous system |
| CT | computed tomography |
| DLT | dose-limiting toxicity |
| EC | ethics committee |
| ECD | extracellular domain |
| ECG | electrocardiogram |
| ECHO | echocardiogram |
| eCRF | electronic Case Report Form |
| EDC | electronic data capture |
| FACT-B | Functional Assessment of Cancer |
| | Therapy-Breast |
| FDA | Food and Drug Administration |
| FISH | fluorescence in situ hybridization |
| G-CSF | granulocyte colony-stimulating factor |
| GM-CSF | granulocyte macrophage |
| | colony-stimulating factor |
| HER2 | human epidermal growth factor receptor 2 |
| ICH | International Conference on Harmonisation |
| IHC | immunohistochemistry |
| IND | Investigational New Drug |
| IRB | institutional review board |
| IRF | independent review facility |
| IVRS | interactive voice response system |
| LS | liquid chromatography |
| MS | mass spectrometry |
| LVEF | left ventricular ejection fraction |
| MBC | metastatic breast cancer |
| MRI | magnetic resonance imaging |
| MTD | maximum tolerated dose |
| MUGA | multiple-gated acquisition |
| NCI CTCAE | National Cancer Institute Common |
| | Terminology Criteria for Adverse Events |
| NYHA | New York Heart Association |
| PFS | progression-free survival |
| PK | pharmacokinetic |
| PR | partial response |
| RECIST | Response Evaluation Criteria in Solid |
| | Tumors |
| RR | response rate |
| RT-PCR | reverse transcriptase polymerase chain |
| | reaction |
| SAE | serious adverse event |
| SD | stable disease |
| SWFI | Sterile Water for Injection |
| T-DM1 | trastuzumab-MCC-DM1 |
| TOI-PFB | Trial Outcomes Index-Physical |
| | Functional Breast |
| ULN | upper limit of normal |
| VAS | visual analog scale |

TRASTUZUMAB-MCC-DM1

The present invention includes therapeutic treatments with trastuzumab-MCC-DM1 (T-DM1), an antibody-drug conjugate (CAS Reg. No. 139504-50-0), which has the structure:

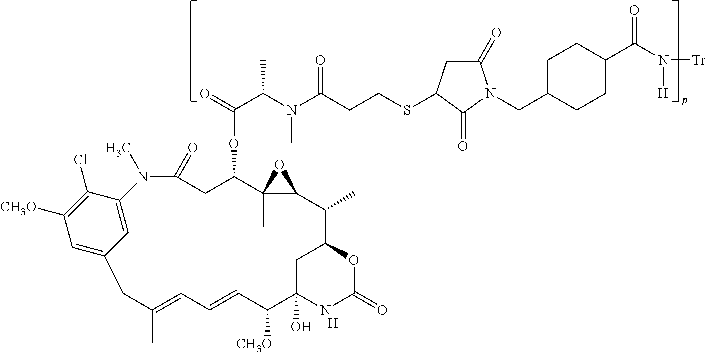

where Tr is trastuzumab linked through linker moiety MCC to the maytansinoid drug moiety DM1 (U.S. Pat. No. 5,208,020; U.S. Pat. No. 6,441,163). The drug to antibody ratio or drug loading is represented by p in the above structure of trastuzumab-MCC-DM1, and ranges in integer values from 1 to about 8. Trastuzumab-MCC-DM1 includes all mixtures of variously loaded and attached antibody-drug conjugates where 1, 2, 3, 4, 5, 6, 7, and 8 drug moieties are covalently attached to the antibody trastuzumab (U.S. Pat. No. 7,097,840; US 2005/0276812; US 2005/0166993). Trastuzumab-MCC-DM1 may be prepared according to Example 1.

Trastuzumab is produced by a mammalian cell (Chinese Hamster Ovary, CHO) suspension culture. The HER2 (or c-erbB2) proto-oncogene encodes a transmembrane receptor protein of 185 kDa, which is structurally related to the epidermal growth factor receptor. Trastuzumab is an antibody that has antigen binding residues of, or derived from, the murine 4D5 antibody (ATCC CRL 10463, deposited with American Type Culture Collection, 12301 Parklawn Drive, Rockville, Md. 20852 under the Budapest Treaty on May 24, 1990). Exemplary humanized 4D5 antibodies include huMAb4D5-1, huMAb4D5-2, huMAb4D5-3, huMAb4D5-4, huMAb4D5-5, huMAb4D5-6, huMAb4D5-7 and huMAb4D5-8 (HERCEPTIN®) as in U.S. Pat. No. 5,821,337.

Prior Treatment History

Patients treated with T-DM1 by the methods of the invention were previously treated with trastuzumab, lapatinib, capecitabine, an anthracycline, and a taxane.

Taxanes include but are not limited to docetaxel, paclitaxel, larotaxel (CAS Reg. No. 156294-36-9), tesetaxel (CAS Reg. No. 333754-36-2), and ortataxel (CAS Reg. No. 186348-23-2).

Anthracyclines include but are not limited to doxorubicin, daunorubicin (daunomycin, CERUBIDINE®, CAS Reg. No. 20830-81-3), epirubicin (ELLENCE®, Pfizer, CAS Reg. No. 56420-45-2, idarubicin (4-demethoxydaunorubicin, ZAVEDOS® (UK); IDAMYCIN®, CAS Reg. No. 58957-92-9), valrubicin (VALSTAR®, Endo Pharmaceuticals, CAS Reg. No. 56124-62-0), mitoxantrone (1,4-dihydroxy-5,8-bis[2-(2-hydroxyethylamino)ethylamino]-anthracene-9,10-dione, CAS Reg. No. 65271-80-9), and pixantrone (6,9-bis[(2-aminoethyl)amino]benzo[g]isoquinoline-5,10-dione, CAS Reg. No. 144510-96-3).

Trastuzumab (HERCEPTIN®, Genentech, Inc., CAS Reg. No. 180288-69-1) is a recombinant, humanized monoclonal antibody (IgG1 isotype) directed against the extracellular region of HER2 that has been developed as a therapeutic modality for treating HER2-positive breast cancer. Trastuzumab is an anti-HER2 agent. Trastuzumab is approved by the U.S. Food and Drug Administration (FDA) for the adjuvant treatment of patients with HER2-overexpressing, node-positive breast cancer as part of a treatment regimen containing doxorubicin, cyclophosphamide, and paclitaxel. Trastuzumab is also indicated as a single agent and in combination with paclitaxel for the treatment of patients with HER2-positive metastatic breast cancer (MBC).

Lapatinib (CAS Reg. No. 388082-78-8, TYKERB®, GW572016, Glaxo SmithKline) has been approved for use in combination with capecitabine (XELODA®, Roche) for the treatment of patients with advanced or metastatic breast cancer whose tumors over-express HER2 (ErbB2) and who have received prior therapy including an anthracycline, a taxane and trastuzumab. Lapatinib is an anti-HER2 agent. Lapatinib is an ATP-competitive epidermal growth factor (EGFR) and HER2/neu (ErbB-2) dual tyrosine kinase inhibitor (U.S. Pat. No. 6,727,256; U.S. Pat. No. 6,713,485; U.S. Pat. No. 7,109,333; U.S. Pat. No. 6,933,299; U.S. Pat. No. 7,084,147; U.S. Pat. No. 7,157,466; U.S. Pat. No. 7,141,576) which inhibits receptor autophosphorylation and activation by binding to the ATP-binding pocket of the EGFR/HER2 protein kinase domain. Lapatinib is named as N-(3-chloro-4-(3-fluorobenzyloxyl)phenyl)-6-(5-((2-(methylsulfonyl)ethylamino)methyl)furan-2-yl)quinazolin-4-amine, and has the structure:

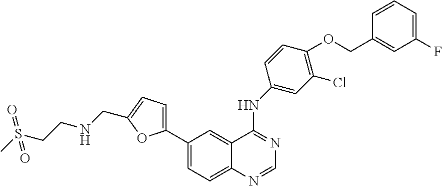 [SMILES: CS(=O)(=O)CCNCc1ccc(-c2ccc3ncnc(Nc4ccc(OCc5cccc(F)c5)c(Cl)c4)c3c2)o1]

Capecitabine (CAS Reg. No. 154361-50-9, XELODA®, Roche) is an orally-administered chemotherapeutic agent used in the treatment of metastatic breast and colorectal cancers. Capecitabine may be named as pentyl[1-(3,4-dihydroxy-5-methyl-tetrahydrofuran-2-yl)-5-fluoro-2-oxo-1H-pyrimidin-4-yl]aminomethanoate or pentyl 1-((3R,4S,5R)-3,4-dihydroxy-5-methyltetrahydrofuran-2-yl)-5-fluoro-2-oxo-1,2-dihydropyrimidin-4-ylcarbamate; and has the structure:

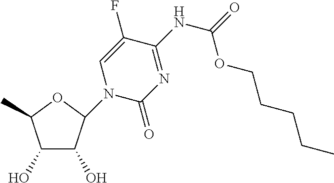 [SMILES: CCCCCOC(=O)Nc1nc(=O)n(C2O[C@H](C)[C@@H](O)[C@H]2O)cc1F]

Doxorubicin (ADRIAMYCIN®, hydroxyldaunorubicin) is a DNA-interacting drug widely used in chemotherapy since the 1960s. It is an anthracycline antibiotic and structurally related to daunomycin, which also intercalates DNA. Doxorubicin is commonly used in the treatment of a wide range of cancers. Doxorubicin is named as (8S,10S)-10-(4-amino-5-hydroxy-6-methyl-tetrahydro-2H-pyran-2-yloxy)-6,8,11-trihydroxy-8-(2-hydroxyacetyl)-1-methoxy-7,8,9,10-tetrahydrotetracene-5,12-dione, (CAS Reg. No. 23214-92-8) and has the structure:

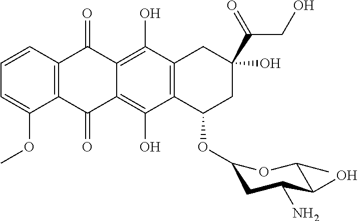

Docetaxel (TAXOTERE®, Sanofi-Aventis) is used to treat breast, ovarian, and NSCLC cancers (U.S. Pat. No. 4,814,470; U.S. Pat. No. 5,438,072; U.S. Pat. No. 5,698,582; U.S. Pat. No. 5,714,512; U.S. Pat. No. 5,750,561). Docetaxel is named as (2R,3S)—N-carboxy-3-phenylisoserine, N-tert-butyl ester, 13-ester with 5,20-epoxy-1,2,4,7,10,13-hexahydroxytax-11-en-9-one 4-acetate 2-benzoate, trihydrate (U.S. Pat. No. 4,814,470; EP 253738; CAS Reg. No. 114977-28-5) and has the structure:

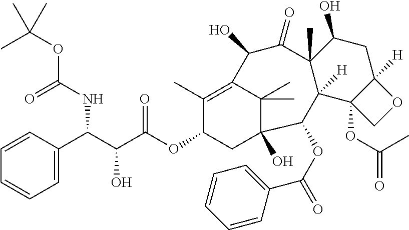

Paclitaxel (TAXOL®, Bristol-Myers Squibb Oncology, Princeton N.J., CAS Reg. No. 33069-62-4) is a compound isolated from the bark of the Pacific yew tree, Taxus brevifolia, and used to treat lung, ovarian, breast cancer, and advanced forms of Kaposi's sarcoma (Wani et al (1971) J. Am. Chem. Soc. 93:2325; Mekhail et al (2002) Expert. Opin. Pharmacother. 3:755-766). Paclitaxel is named as β-(benzoylamino)-α-hydroxy-,6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca(3,4)benz(1,2-b) oxet-9-ylester,(2aR-(2a-α,4-β,4a-β,6-β,9-α (α-R*,β-S*),11-α,12-α,12a-α,2b-α))-benzenepropanoic acid, and has the structure:

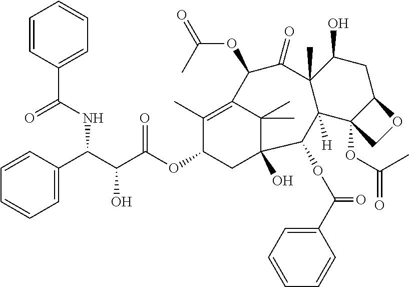 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Clinical Study Objectives

This Phase II study evaluated the safety and efficacy (as evidenced by objective response rate and duration of objective response) of T-DM1 in HER2-positive metastatic breast cancer (MBC) patients who have received prior treatment, as described below.

The primary objectives of the Phase II clinical study were to assess the objective response rate (through independent radiologic review) of HER2-positive MBC patients treated with T-DM1 and to characterize the safety and tolerability of T-DM1 in this patient population. The secondary objectives of this study were to further characterize the efficacy of T-DM1 in this patient population, as measured by duration of objective response, clinical benefit rate (CBR), which is the proportion of patients with CR, PR, and SD at 6 months, and progression-free survival (PFS), based on independent radiologic review and to characterize the pharmacokinetics of T-DM1 in this patient population.

The exploratory objectives of this study were to: (i) investigate whether the level of HER2 gene amplification (as assessed on fluorescence in situ hybridization [FISH]) and/or mRNA expression (as assessed on reverse transcriptase polymerase chain reaction [RT-PCR]) in archival tumor tissue correlates with T-DM1 efficacy; (ii) to investigate whether levels of expression of HER family receptors (including altered forms of HER2) and/or ligands in archival tumor tissue correlate with T-DM1 efficacy; (iii) conduct an exploratory exposure-effect analysis to investigate the relationship between the pharmacokinetics of T-DM1 and drug effect (e.g., efficacy, safety); (iv) investigate whether Fcγ-receptor polymorphisms correlate with T-DM1 efficacy; (v) investigate the potential effect of FcγRIIa and 131 tubulin polymorphism status on the incidence and severity of thrombocytopenia; (vi) assess the formation of antibodies to T-DM1; (vii) compare the differences in symptoms between clinical responders and non-responders using the Trial Outcomes Index-Physical Functional Breast (TOI-PFB) subscale of the Functional Assessment of Cancer Therapy-Breast (FACT-B) instrument and Patient's Assessment of Pain; (viii) measure pain intensity within the previous 24 hours, using a numeric rating scale (Dworkin R H, Turk D C, Farrar J T, et al. Core outcome measures for chronic pain clinical trials: IMMPACT recommendations. Pain 2005; 113:9-19) with a visual analog scale (VAS) hashed line; (ix) investigate cardiac troponin I as a prognostic marker for heart failure for patients treated with T-DM1; and (x) explore the safety of T-DM1 when used in patients who have developed isolated brain metastases while on study treatment.

The study was a Phase II, multi-institutional, single-arm, open-label study of T-DM1 administered at a dose of 3.6 mg/kg of patient body weight by IV infusion every 3 weeks to patients with HER2-positive MBC. A total of approximately 110 patients were enrolled and treated, and the total enrollment period was approximately 50 weeks. The analysis of objective response rate was conducted with patient data collected through approximately 26 weeks after the last patient is enrolled in the study. This study enrolled patients with HER2-positive MBC. Prior treatment included an anthracycline, trastuzumab, a taxane, lapatinib, and capecitabine given in the neoadjuvant, adjuvant, or metastatic setting, or as treatment for locally advanced disease. Patients must have been treated with two or more regimens in the metastatic or locally advanced setting and have progressed on their most recent treatment. Patients must have been treated with at least two anti-HER2 agents (also referred to as “HER2-targeted agents”) in the metastatic setting or unresectable locally advanced setting. The HER2-targeted agents must have included trastuzumab and lapatinib. Patients must have progressed on their most recent treatment. Patients will be required to have HER2-positive status, as evidenced by either 3+HER2 protein overexpression determined by immunohistochemistry (IHC) or HER2 gene amplification determined by FISH.

Efficacy Assessments: Tumor responses were categorized as complete response (CR), partial response (PR), stable disease (SD), or progressive disease (PD) according to the Response Evaluation Criteria for Solid Tumors (RECIST). Tumor assessments (CT and/or magnetic resonance imaging [MRI] scans) were performed approximately every 6 weeks irrespective of dose delays, interruptions, or reductions. Bone and brain scans (either CT or MRI) were performed at baseline and if clinically indicated during the study. Patient management decisions were made based on tumor assessments performed by investigators. The primary study endpoints related to response were determined by an independent radiologic review of patient scans, with assessments based on investigator reporting being secondary. If each non-target lesion cannot be assessed at a follow-up tumor assessment timepoint, patients may still be considered evaluable for timepoint response as long as all target lesions are measured. Patients with non-target lesions that were not assessed at a timepoint were assessed as having a partial response, stable disease, or progressive disease. All lesions, target and non-target, were assessed before a response was considered confirmed. To reduce the frequency of unevaluable non-target lesions, bone lesions visible on baseline CT scan as non-target lesion(s) for follow-up, were compared with bone lesions identified on the baseline bone scan where possible if lesions are not visible on both modalities or as easily and reproducibly assessed.

Safety, pharmacokinetics, and diagnostic assessments were made in all treated patients.

Patient Group Selection: Nonclinical studies indicate that the sensitivity of cancer cells to T-DM1 requires HER2 overexpression, and measurement of such expression is a standard of care in determining eligibility for trastuzumab therapy (HERCEPTIN® Package Insert). Measurement of HER2 gene amplification has been performed using IHC measurement. HER2 gene amplification has also proven to be a reliable method for demonstrating HER2-positive status (Persons D L, Bui M M, Lowery M C, et al. Fluorescence in situ hybridization (FISH) for detection of HER-2/neu amplification in breast cancer: a multicenter portability study. (2000) Ann Clin Lab Sci 30:41-8). Patients with MBC whose tumors are positive for HER2 overexpression are most likely to benefit from T-DM1 and represent the patient population eligible for study enrollment.

Continued Dosing: Several patients received 12+ months of treatment with T-DM1 at the MTD (3.6 mg/kg) on an every-3-week schedule (the regimen to be used in the current study), and some are nearing 2 years of treatment. Patients who meet the criteria for ongoing clinical benefit may be allowed to continue study treatment in the absence of disease progression (except for isolated brain metastases under certain conditions), unacceptable toxicity, or until study closure. An extension study is available for those patients who continue to receive benefit from T-DM1 after study closure.

Efficacy Assessments: An assessment of the objective response rate, the primary objective of this trial, was based on the RECIST and confirmed by independent review of baseline and follow-up assessments obtained every 6 weeks. All known sites of disease should be reassessed at each timepoint whenever possible. Because bone involvement is a common finding in patients with MBC, a bone scan at baseline may be conducted. In addition, patients with non-target lesions that were not assessed at a timepoint were assessed as having a partial response, stable disease, or progressive disease. All lesions, target and non-target (including bone lesions), were assessed before a complete response can be considered confirmed.

Exploratory Diagnostic Studies: Current determination of HER2 status in breast cancer is made according to a semi-quantitative algorithm with a binary result (positive or negative, amplified or non-amplified). An open question remains as to whether levels of HER2 mRNA expression or gene amplification within the positive range are correlated with degree of response to HER2-targeted therapies. Patients were classified into quartiles based on the degree of HER2 gene amplification and level of mRNA expression to assess the potential relationship between response and these variables. HER2 signaling is known to be modulated by expression levels of other HER family members (e.g., HER1 and HER3) and ligands (e.g., amphiregulin, betacellulin, neuregulin). Several studies have indicated that expression of these genes may correlate with response or resistance to HER2-targeted therapies (Wiseman S M, Makretsov N, Nielsen T O, et al. Coexpression of the type 1 growth factor receptor family members HER-1, HER-2, and HER-3 has a synergistic negative prognostic effect on breast carcinoma survival. (2005) Cancer 103: 1770-7; Robinson A G, Turbin D, Thomson T. Molecular predictive factors in patients receiving trastuzumab-based chemotherapy for metastatic disease. (2006) Clin Breast Cancer 7:254-61).

Pharmacogenomic Studies: The Phase II clinical plan was designed to further explore whether FcγR polymorphisms are associated with efficacy on trastuzumab-DM1.

Safety Monitoring Plan: The safety plan addresses the anticipated toxicities of T-DM1 based on clinical and nonclinical testing, clinical testing of its components (trastuzumab and DM1), and clinical testing of other DM1-containing ADCs.

Pharmacokinetic Assessment Plan: The combination of data obtained in this study (and in the Phase I and ongoing Phase II studies) allowed complete profiling of the distribution and elimination phases for this molecule and the investigation of potential correlations between various PK parameters and efficacy and/or toxicity. New clinical material is likely to be introduced in this study; thus, exploratory comparisons across studies may be done to verify the comparability of the PK profile. In addition to pre-dose, post-dose, and weekly samples in Cycles 1 through 4, PK samples will be obtained every other cycle after Cycle 4, with the exception of Cycles 10 and 14. Serum concentration—time data collected throughout treatment in this study combined with data from previous Phase I and Phase II studies can be modeled to estimate population PK parameters (mean and inter-patient variability) as well as the relationship between T-DM1 CL and pathophysiologic covariates. The presence and/or levels of soluble HER2 extracellular domain (ECD) at baseline may affect T-DM1 PK parameters. Therefore, HER2 ECD levels in serum will be assessed only at baseline.

Assessment of Patient-Reported Outcomes: TOI-PFB score data and Patient's Assessment of Pain results collected in this single-arm study will provide a preliminary characterization of the differences in symptoms among clinically responding and non-responding patients associated with T-DM1 treatment in this patient population. These data will be used to help evaluate the feasibility of testing symptom-progression hypotheses in future randomized trials.

Continuing Treatment Post-Progression in the Brain: In clinical practice, clinicians typically will continue trastuzumab, with or without chemotherapy, in HER2-positive women who have developed brain disease but have demonstrated control of their visceral disease. A retrospective study demonstrated that the median survival of patients with HER2-positive disease treated with trastuzumab after the diagnosis of brain metastasis was 11.9 months, significantly longer than those with HER2-positive disease who did not receive trastuzumab after the diagnosis of CNS disease (3.0 months, p=0.05) (Church D N, Modqil R, Guglani S, et al. Extended survival in women with brain metastases from HER2 overexpressing breast cancer. (2008) Am J Clin Oncol 31:250-4). In fact, the availability of trastuzumab has been hypothesized as one of the possible reasons why women with HER2-overexpressing MBC appear to have significantly longer survival compared with women with HER2-negative disease after the diagnosis of brain metastases (Church D N, Modqil R, Guglani S, et al. (2008) Am J Clin Oncol 31:250-4). By allowing T-DM1 treatment after an isolated progression in the brain has been identified and treated, this study will evaluate whether T-DM1 is safe in this patient population.

Efficacy Outcome Measures: The primary efficacy outcome measure is objective response (defined as a complete or partial response determined on two consecutive occasions ≧4 weeks apart), as assessed through independent radiologic review using RECIST. The secondary efficacy outcome measures are as follows: Duration of objective response, as assessed through independent radiologic review using RECIST; PFS, as assessed through independent radiologic review using RECIST; CBR based on independent radiologic review using RECIST; Objective response based on investigator assessments using RECIST; Duration of objective response based on investigator assessments using RECIST; PFS based on investigator assessments using RECIST; and CBR based on investigator assessments using RECIST.

Safety Outcome Measures are as follows: Incidence of adverse events and serious adverse events; Incidence, nature, and relatedness of serious adverse events; Incidence of adverse events leading to T-DM1 discontinuation, modification, or interruption; Incidence and magnitude of declines in LVEF; incidence of symptomatic CHF; and Cause of death on study.

Pharmacokinetic Outcome Measures are as follows: Serum concentrations of total trastuzumab and T-DM1; and Plasma concentrations of free DM1.

The exploratory outcome measures are as follows: Change in patient-reported symptoms from baseline to each timepoint as assessed by the FACT-B subscale (TOI-PFB) and Patient's Assessment of Pain for the following cohorts: all patients treated with T-DM1, clinical responders, and patients with stable disease or clinical non-responders; Levels of HER2 gene amplification and mRNA expression in archival tumor tissue; Expression of HER family receptors (other than HER2) and ligands in archival tumor tissue; Exposure-effect analysis to investigate the relationship between the pharmacokinetics of T-DM1 and drug effect (e.g., efficacy, safety); FcγR polymorphism status (FcγRIIa, FcγRIIc, and FcγRIIIa); β1 Tubulin polymorphism status; Formation of antibodies to T-DM1; Cardiac troponin I as a prognostic marker for heart failure in patients treated with T-DM1; and Incidence of adverse events, serious adverse events, and cause of death in patients who develop isolated and treatable brain metastases.

### CLINICAL STUDY STATUS

### Methods of Treatment

A Phase II, single-arm, open-label study of trastuzumab-MCC-DM1 administered intravenously to patients with HER2-positive metastatic breast cancer was conducted. The primary objectives of this study were to assess the objective response rate (ORR) through independent radiologic review of HER2-positive metastatic breast cancer (MBC) patients treated with T-DM1. The secondary objectives were to further characterize the efficacy of T-DM1 in this patient population, as measured by duration of objective response, clinical benefit rate (CBR), which is the proportion of patients with complete response (CR), partial response (PR), and stable disease (SD) at 6 months, and progression-free survival (PFS), based on independent radiologic review.

The clinical study concluded with 110 patients receiving at least one dose of T-DM1, 91 of which had central HER2 data available. Of those, 76 (83.5%) were confirmed HER2+ retrospectively. Further baseline characteristics of the patient population include those in Table 1 in Example 4.

Median follow-up is 8.3 months (range 0.7-13.1). As of the data cutoff date, 40 patients were on study. Of the 70 patients that discontinued treatment: 58 had progressive disease (52.7%) and 6 had one or more adverse events (5.5%).

Also, 109 of the 110 patients had received prior trastuzumab, capecitabine, anthracycline, taxane and lapatinib. The prior chemotherapy and anti-HER2 therapy data of the patient population includes those in Table 2 in Example 4.

The pharmacokinetics (PK) parameters for T-DM1 conjugate in this phase II study were similar to those reported at the MTD in the phase I study, and to PK parameters for the previously reported 112 patient proof-of-concept Phase II Study. Maximum serum T-DM1 concentration (Cₘₐₓ, μg/ml=76.8, terminal halflife in days (t½)=4.0, Clearance (CL, ml/day/kg)=8.3. Patient exposure to drug data for the 110 patients included those in Table 3 in Example 4.

Antitumor Activity

Primary analysis of the antitumor activity data included that in Table 4, Example 7.

The antitumor activity in treated HER2 normal and HER2 positive patients by retrospectively confirmed HER2 status includes that in Table 5, Example 7.

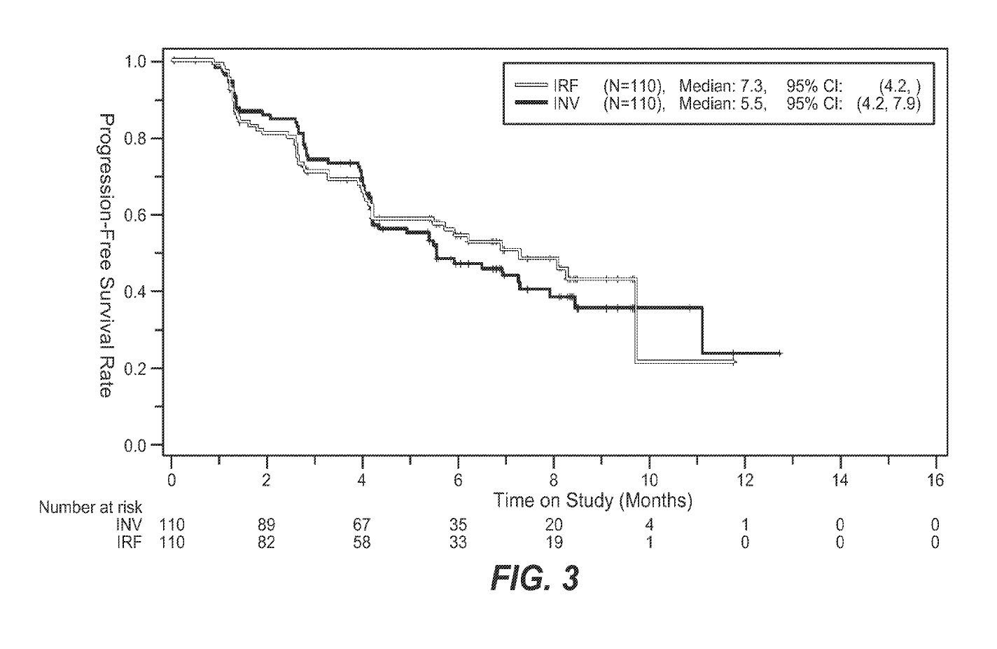

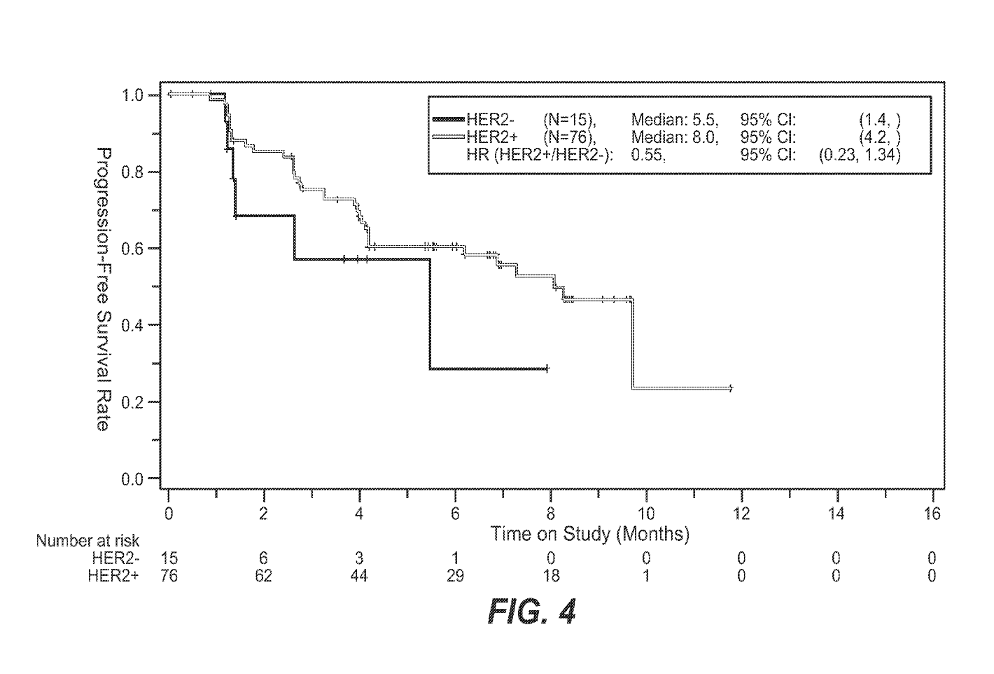

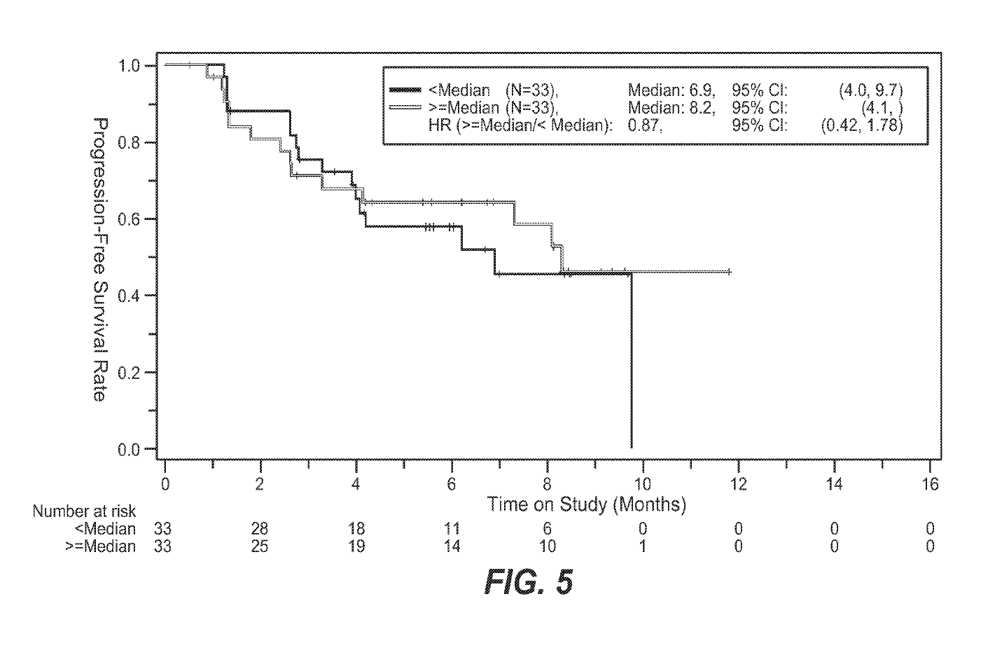

FIG. 3 shows a Kaplan-Meier plot of progression-free survival (PFS) in all treated patients. An updated analysis yielded similar results, with median PFS of 6.9 (IRF) and 95% CI (4.2-8.4). FIG. 4 shows a Kaplan-Meier plot of progression-free survival (PFS) in patients separated by HER2 status. FIG. 5 shows a Kaplan-Meier plot of PFS per IRF Assessment by HER2 qRT-PCR for Retrospectively Confirmed HER2+ treated patients.

A summary of IRF and investigator (INV) assessment includes those in Table 6 in Example 7. The ORR endpoint of the Clinical Study was 32.7% IRF, 30% INV, and CBR of 44.5% IRF, 40% INV. Anti-tumor activity was seen in the predefined patient population which was previously treated with an anthracycline, a taxane, capecitabine, trastuzumab, and lapatinib. Patients on study received two HER2-directed regimens in the metastatic setting. Progressive disease on last regimen received. This specific population has not been previously studied. T-DM1 is well tolerated by patients at the dose and schedule tested with no dose-limiting cardiotoxicity or new safety signals. One patient died from hepatic dysfunction. The toxicities observed on this study are acceptable and manageable in this patient population.

### FORMULATIONS

Trastuzumab-MCC-DM1 may be formulated in accordance with standard pharmaceutical practice for use in a therapeutic combination for therapeutic treatment (including prophylactic treatment) of hyperproliferative disorders in mammals including humans. The invention provides a pharmaceutical composition comprising trastuzumab-MCC-DM1 in association with one or more pharmaceutically acceptable carrier, glidant, diluent, or excipient.

Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration with pharmaceutically acceptable diluents, carriers, excipients or stabilizers (Remington's Pharmaceutical Sciences (1995) 18th edition, Mack Publ. Co., Easton, Pa.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8.

The pharmaceutical formulation is preferably sterile. In particular, formulations to be used for in vivo administration must be sterile. Such sterilization is readily accomplished by filtration through sterile filtration membranes.

The pharmaceutical formulation ordinarily can be stored as a solid composition, a lyophilized formulation or as an aqueous solution.

The pharmaceutical formulations of the invention will be dosed and administered in a fashion, i.e., amounts, concentrations, schedules, course, vehicles and route of administration, consistent with good medical practice. Factors for consideration in this context include the particular disorder being treated, the clinical condition of the individual patient, the cause of the disorder, the site of delivery of the agent, the method of administration, the scheduling of administration, and other factors known to medical practitioners.

As a general proposition, the initial pharmaceutically effective amount of trastuzumab-MCC-DM1 administered per dose will be in the range of about 0.3 to 15 mg/kg/day of patient body weight.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, ethanol, or benzylalcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, including Tween 80, PLURONICS™ or polyethylene glycol (PEG), including PEG400. The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, (1995) Mack Publ. Co., Easton, Pa. Other examples of drug formulations can be found in Liberman, H. A. and Lachman, L., Eds., Pharmaceutical Dosage Forms, Marcel Decker, Vol 3, 2^{nd} Ed., New York, N.Y.

The pharmaceutical formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences 18^{th} Ed. (1995) Mack Publishing Co., Easton, Pa. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may be a solution or a suspension in a non-toxic parenterally acceptable diluent or solvent, such as a solution in 1,3-butanediol or prepared from a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

Combination Therapy

Trastuzumab-MCC-DM1 may be employed in combination with other chemotherapeutic agents for the treatment of a hyperproliferative disease or disorder, including tumors, cancers, and neoplastic tissue, along with pre-malignant and non-neoplastic or non-malignant hyperproliferative disorders. In certain embodiments, trastuzumab-MCC-DM1 is combined in a pharmaceutical combination formulation, or dosing regimen as combination therapy, with a second compound that has anti-hyperproliferative properties or that is useful for treating the hyperproliferative disorder. The second compound of the pharmaceutical combination formulation or dosing regimen preferably has complementary activities to trastuzumab-MCC-DM1, and such that they do not adversely affect each other. Such compounds are suitably present in combination in amounts that are effective for the purpose intended. In one embodiment, a composition of this invention comprises trastuzumab-MCC-DM1 in combination with a chemotherapeutic agent such as described herein.

Therapeutic combinations of the invention include a formulation, dosing regimen, or other course of treatment comprising the administration of trastuzumab-MCC-DM1, and a chemotherapeutic agent selected from a HER2 dimerization inhibitor antibody, an anti-VEGF antibody, 5-FU, carboplatin, lapatinib, ABT-869, and docetaxel, as a combined preparation for separate, simultaneous or sequential use in the treatment of a hyperproliferative disorder.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments.

In a particular embodiment of anti-cancer therapy, trastuzumab-MCC-DM1 may be combined with a chemotherapeutic agent, including hormonal or antibody agents such as those described herein, as well as combined with surgical therapy and radiotherapy. The amounts of trastuzumab-MCC-DM1 and the other pharmaceutically active chemotherapeutic agent(s) and the relative timings of administration will be selected in order to achieve the desired combined therapeutic effect.

Administration of T-DM1

Pharmaceutical compositions of trastuzumab-MCC-DM1 (T-DM1) may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Topical administration can also involve the use of transdermal administration such as transdermal patches or iontophoresis devices. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier, glidant, or excipient. Where the compound is administered parenterally, it may be formulated with a pharmaceutically acceptable parenteral vehicle or diluent, and in a unit dosage injectable form, as detailed below.

A dose of trastuzumab-MCC-DM1 to treat human patients may range from about 100 mg to about 500 mg. The dose of trastuzumab-MCC-DM1 may be administered once every six weeks, once every three weeks, weekly, or more frequently, depending on the pharmacokinetic (PK) and pharmacodynamic (PD) properties, including absorption, distribution, metabolism, and excretion. A dose of the chemotherapeutic agent, if used in combination with trastuzumab-MCC-DM1, may range from about 10 mg to about 1000 mg. The chemotherapeutic agent may be administered once every six weeks, once every three weeks, weekly, or more frequently, such as once or twice per day. In addition, toxicity factors may influence the dosage and administration regimen. When administered orally, the pill, capsule, or tablet may be ingested daily or less frequently for a specified period of time. The regimen may be repeated for a number of cycles of therapy.

Articles of Manufacture

In another embodiment of the invention, an article of manufacture, or “kit”, containing trastuzumab-MCC-DM1 useful for the treatment of the diseases and disorders described above is provided. In one embodiment, the kit comprises a container comprising trastuzumab-MCC-DM1. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold trastuzumab-MCC-DM1 or a formulation thereof which is effective for treating the condition and may have a sterile access port (for example, the container may be an intravenous solution bag or a vial having a stopper pierceable by a hypodermic injection needle). At least one active agent in the composition is trastuzumab-MCC-DM1, which may be in lyophilized form. The label or package insert indicates that the composition is used for treating the condition of choice, such as cancer. In one embodiment, the label or package inserts indicates that the composition comprising trastuzumab-MCC-DM1 can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of trastuzumab-MCC-DM1 and, if present, the second pharmaceutical formulation. For example, if the kit comprises a first composition comprising trastuzumab-MCC-DM1 and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of trastuzumab-MCC-DM1, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with trastuzumab-MCC-DM1 contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Where the kit comprises a composition of trastuzumab-MCC-DM1 and a second therapeutic agent, i.e. the chemotherapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### EXAMPLES

In order to illustrate the invention, the following examples are included. However, it is to be understood that these examples do not limit the invention and are only meant to suggest a method of practicing the invention.

### Example 1

### Preparation of Trastuzumab-MCC-DM1

Trastuzumab was purified from HERCEPTIN® by buffer-exchange at 20 mg/mL in 50 mM potassium phosphate/50 mM sodium chloride/2 mM EDTA, pH 6.5 and treated with 7.5 to 10 molar equivalents of succinimidyl 4-(N-maleimidomethyl)cyclohexane-1-carboxylate (SMCC, Pierce Biotechnology, Inc), 20 mM in DMSO or DMA (dimethylacetamide), 6.7 mg/mL (US 2005/0169933; US 2005/0276812). After stirring for 2 to 4 hours under argon at ambient temperature, the reaction mixture was filtered through a Sephadex G25 column equilibrated with 50 mM potassium phosphate/50 mM sodium chloride/2 mM EDTA, pH 6.5. Alternatively, the reaction mixture was gel filtered with 30 mM citrate and 150 mM sodium chloride at pH 6. Antibody containing fractions were pooled and assayed. Recovery of trastuzumab-SMCC was 88%.

The drug-linker intermediate, trastuzumab-MCC from above, was diluted with 50 mM potassium phosphate/50 mM sodium chloride/2 mM EDTA, pH 6.5, to a final concentration of 10 mg/ml, and reacted with a 10 mM solution of DM1 (1.7 equivalents assuming 5 SMCC/trastuzumab, 7.37 mg/ml) in dimethylacetamide. DM1 may be prepared from ansamitocin fermentation products (U.S. Pat. No. 6,790,954; U.S. Pat. No. 7,432,088) and derivatized for conjugation (U.S. Pat. No. 6,333,410; RE 39151). The reaction was stirred at ambient temperature under argon for 4 to about 16 hours. The conjugation reaction mixture was filtered through a Sephadex G25 gel filtration column (1.5×4.9 cm) with 1×PBS at pH 6.5. Alternatively, the reaction mixture was gel filtered with 10 mM succinate and 150 mM sodium chloride at pH 5. The DM1/trastuzumab ratio (p) was 3.1, as measured by the absorbance at 252 nm and at 280 nm. The drug to antibody ratio (p) may also be measured by mass spectrometry. Conjugation may also be monitored by SDS polyacrylamide gel electrophoresis. Aggregation may be assessed by laser light scattering analysis.

Alternatively, trastuzumab-MCC-DM1 may be prepared by forming an MCC-DM1 linker-drug reagent and then reacting with trastuzumab.

Typically a conjugation reaction of trastuzumab-MCC with DM1 results in a heterogeneous mixture comprising antibodies with different numbers of attached, conjugated DM1 drugs, i.e. drug loading where p is a distribution from 1 to about 8. An additional dimension of heterogeneity exists with different attachment sites of SMCC to trastuzumab where many different nucleophiles on trastuzumab, e.g. terminal lysine amino groups, can react with SMCC. Thus, trastuzumab-MCC-DM1 includes isolated, purified species molecules as well as mixtures of average drug loading from 1 to 8 and where MCC-DM1 is attached through any site of the trastuzumab antibody.

The average number of DM1 drug moieties per trastuzumab antibody in preparations of trastuzumab-MCC-DM1 from conjugation reactions may be characterized by conventional means such as mass spectroscopy, ELISA assay, electrophoresis, and HPLC. The quantitative distribution of trastuzumab-MCC-DM1 in terms of p may also be determined. By ELISA, the averaged value of p in a particular preparation of ADC may be determined (Hamblett et al (2004) Clinical Cancer Res. 10:7063-7070; Sanderson et al (2005) Clinical Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous trastuzumab-MCC-DM1 where p is a certain value from trastuzumab-MCC-DM1 with other drug loadings may be achieved by means such as reverse phase HPLC or electrophoresis.

### Example 2

### T-DM1 Formulation

T-DM1 was provided as a single-use lyophilized formulation in a 20-cc Type I USP (Ph Eur) glass vial fitted with a 20-mm fluoro resin-laminated stopper and aluminum seal with a dark grey flip-off plastic cap. The formulated drug product, after reconstitution with 8.0 mL sterile water for injection (SWFI), contains 20 mg/mL T-DM1, 10 mM sodium succinate, pH 5.0, 6% (w/v) trehalose dihydrate, and 0.02% (w/v) polysorbate 20. Alternatively, 6% (w/v) sucrose may be used instead of 6% (w/v) trehalose dehydrate. Each 20-cc vial contains approximately 172 mg to deliver a vial content of 160 mg of T-DM1. The reconstituted product contains no preservative and is intended for single use only.

Study Drug Preparation of Lyophilized formulation: With a new syringe, add 8.0 mL of SWFI to the vial and swirl gently until completely dissolved. Remove the indicated volume of trastuzumab-MCC-DM1 solution from the vial(s) and add to the IV bag. Gently invert the bag to mix the solution. Parenteral drug products should be inspected visually for particulates and discoloration before administration. To reconstitute, using a new syringe, add 8.0 mL of SWFI to the vial and swirl gently until completely dissolved (do not shake vigorously). Inspect the vial to ensure the product is clear and free of particulates before proceeding. Remove the indicated volume of T-DM1 solution from the vial(s) and add to the IV bag of 250 mL of 0.45% (preferred) or 0.9% normal saline. Gently invert the bag to mix the solution. Please note that if 0.9% saline solution is used, there are additional filtering requirements. When 0.9% sodium chloride is used, the employment of 0.22-μm in-line filters is required. It is also recommended to employ 0.22-μm in-line filters when using 0.45% sodium chloride. Do not shake vigorously.

Trastuzumab-MCC-DM1 Storage after Dilution: The solution of trastuzumab-MCC-DM1 for infusion diluted in polyvinyl chloride (PVC) or latex-free PVC-free polyolefin bags containing 0.9% Sodium Chloride Injection, USP, may be stored at 2° C.-8° C. (36° F.-46° F.) for up to 24 hours prior to use.

### Example 3

### Dosage, Administration, and Storage of Trastuzumab-MCC-DM1

Trastuzumab-MCC-DM1 (T-DM1) was given at a dose of 3.6 mg/kg per patient weight intravenously (IV) every 3 weeks. The total dose will depend on the patient's weight on Day 1 of each cycle. T-DM1 was administered in 21-day cycles. A dose delay of up to 21 days is allowed if needed for resolution of toxicities or other adverse events. If the timing of a protocol-mandated procedure (such as the infusion of T-DM1) coincides with a holiday that precludes the procedure, the procedure is performed on the nearest following date, with subsequent protocol-specified procedures rescheduled accordingly.

Dose Calculation: Trastuzumab-MCC-DM1 is given on the basis of a patient's weight on the day of each infusion.

The initial dose is administered over 90 (±10) minutes. Infusions may be slowed or interrupted for patients experiencing infusion-associated symptoms. Following the initial dose, patients will be observed for at least 90 minutes for fever, chills, or other infusion-associated symptoms. If prior infusions are well tolerated (without any signs or symptoms of infusion reactions), subsequent doses of T-DM1 may be administered over 30 (±10) minutes, with a minimum 30-minute observation period post-infusion. Vital signs should be recorded immediately before, every 15 (±5) minutes during, and 90 (±10) minutes after the first T-DM1 infusion. In subsequent cycles, vital signs should be recorded pre- and post-infusion.

Materials—Dilution solution: sterile normal saline solution for injection (0.9%) Sodium Chloride Injection, USP; Intravenous (IV) flush: sterile normal saline solution (0.9%) Sodium Chloride Injection, USP; IV bag: 250-cc IV bag containing sterile normal saline solution for injection (0.9%) Sodium Chloride Injection, USP; or 250-cc IV bag containing sterile normal saline solution for injection (0.45%) Sodium Chloride Injection, USP; Syringes: 1, 3, 5, 10, 20, 30, and 60 cc; Needles: 20 or 21 gauge; Filter: 0.22-μm Baxter filter or equivalent

Study Drug Administration: The trastuzumab-MCC-DM1 infusion time may be decreased, depending on the patient's tolerability of the infusion. For the first cycle, trastuzumab-MCC-DM1 should be administered as a 90 (±10)-minute IV infusion for the first cycle. If the 90-minute infusion is well tolerated, subsequent infusions may be delivered over 30 (±10) minutes.

### Example 4

### Clinical Study of Trastuzumab-MCC-DM1 (T-DM1) in Treatment of Patients with Metastatic Breast Cancer

A Phase II, single-arm, open-label study of the safety, tolerability, and efficacy of trastuzumab-MCC-DM1 (T-DM1) administered intravenously to patients with HER2-positive metastatic breast cancer who have progressed while receiving prior therapy including treatment with an anthracycline, a taxane, capecitabine, lapatinib, and trastuzumab was designed to meet the Primary Objective of assessing the objective response rate (ORR) through independent radiologic review of HER2-positive metastatic breast cancer (MBC) patients treated with T-DM1; and Second Objectives to further characterize the efficacy of T-DM1 in this patient population, as measured by duration of objective response, clinical benefit rate (CBR), which is the proportion of patients with complete response (CR), partial response (PR), and stable disease (SD) at 6 months, and progression-free survival (PFS), based on independent radiologic review.

Study Design

T-DM1 was administered at a dose of 3.6 mg/kg by intravenous (IV) infusion every 3 weeks to patients with HER2-positive MBC. A total of approximately 110 patients were enrolled and treated, and the total enrollment period was approximately 50 weeks. The analysis of objective response rate was conducted with patient data collected through approximately 26 weeks after the last patient was enrolled in the study. In addition, the final analysis of safety and all secondary and exploratory endpoints will be updated with sufficient data when available (estimated to be approximately 10 months after the last patient is enrolled in the study). After the final analysis, the study was closed. An extension study is available for those patients who continue to receive benefit from T-DM1 after study closure. This study enrolled patients with HER2-positive MBC. Prior treatment included an anthracycline, trastuzumab, a taxane, lapatinib, and capecitabine given in the neoadjuvant, adjuvant, or metastatic setting, or as treatment for locally advanced disease. Patients were previously treated with two or more regimens in the metastatic or locally advanced setting and have progressed on their most recent treatment. Patients must have been previously treated with at least two anti-HER2 agents in the metastatic setting or unresectable locally advanced setting; the HER2-targeted agents must have included trastuzumab and lapatinib; and they must have progressed on their most recent treatment. Patients have HER2-positive status, as evidenced by either 3+HER2 protein overexpression determined by immunohistochemistry or HER2 gene amplification determined by fluorescence in situ hybridization.

Patients may remain on study treatment until documented disease progression (with the possible exception of isolated central nervous system [CNS] progression) or unmanageable toxicity, or study termination. An extension study is available for those patients who continue to receive benefit from T-DM1 after study closure

Outcome Measures

The primary efficacy outcome measure is objective response (defined as a complete or partial response determined on two consecutive occasions ≧4 weeks apart), as assessed through independent radiologic review using Response Evaluation Criteria for Solid Tumors (RECIST).

The secondary efficacy outcome measures are: Duration of objective response, as assessed through independent radiologic review using RECIST; PFS, as assessed through independent radiologic review using RECIST; CBR based on independent radiologic review using RECIST; Objective response based on investigator assessments using RECIST; Duration of objective response based on investigator assessments using RECIST; PFS based on investigator assessments using RECIST.

The safety outcome measures are: Incidence of adverse events and serious adverse events; Incidence, nature, and relatedness of serious adverse events; Incidence of adverse events leading to T-DM1 discontinuation, modification, or interruption; Incidence and magnitude of declines in left ventricular ejection fraction (LVEF) and incidence of symptomatic congestive heart failure (CHF); Cause of death on study.

The pharmacokinetic outcome measures are: Serum concentrations of total trastuzumab and T-DM1; and Plasma concentrations of free DM1.

Eligibility Inclusion Criteria: Age ≧18 years; Histologically documented breast cancer; HER2-positive disease; Metastatic breast cancer; Disease progression on the last chemotherapy regimen received in the metastatic setting; Prior treatment with an anthracycline, trastuzumab, a taxane, lapatinib, and capecitabine in the neoadjuvant, adjuvant, locally advanced, or metastatic setting and prior treatment with at least two lines of therapy (a line of therapy can be a combination of two agents or single-agent chemotherapy) in the metastatic setting.

In addition, at least two lines of anti-HER2 therapy must have been given in the metastatic setting as monotherapy or combined with chemotherapy or hormonal therapy. The HER2-targeted agent can include trastuzumab, lapatinib, or an investigational agent with HER2-inhibitory activity.

A minimum of 6 weeks of trastuzumab for the treatment of metastatic disease is also required.

Patients must have had at least 14 days of exposure in the metastatic setting to lapatinib and capecitabine (given together or separately) unless they were intolerant of lapatinib and/or capecitabine.

Exclusion Criteria are: Chemotherapy ≦21 days before enrollment; Trastuzumab ≦21 days before enrollment; Hormone therapy ≦7 days before enrollment; Granulocyte-stimulating agent <14 days before enrollment; Investigational therapy ≦28 days before enrollment; Previous radiotherapy for treatment of metastatic breast cancer ≦21 days before enrollment; Brain metastases that are untreated, symptomatic, or require therapy to control symptoms; or any radiation, surgery, or other therapy to control symptoms from brain metastases within 3 months of the first study treatment; History of intolerance (including Grade 3-4 infusion reaction) or hypersensitivity to trastuzumab or murine proteins; History of exposure to the following cumulative doses of anthracyclines: Doxorubicin or liposomal doxorubicin >500 mg/m²; Epirubicin >900 mg/m²; Mitoxantrone >120 mg/m² and idarubicin >90 mg/m²; Peripheral neuropathy of Grade ≧3 per NCI CTCAE, v3.0; History of other malignancy within the last 5 years, except for carcinoma in situ of the cervix or basal cell carcinoma; Current unstable angina; History of symptomatic congestive heart failure (CHF), or ventricular arrhythmia requiring treatment; History of myocardial infarction within 6 months of enrollment; LVEF (left ventricular ejection fraction)<50% within 28 days of enrollment; History of decreased LVEF to <50% or symptomatic CHF with previous adjuvant trastuzumab treatment; Severe dyspnea at rest due to complications of advanced malignancy or requiring current continuous oxygen therapy; Current severe, uncontrolled systemic disease (e.g., clinically significant cardiovascular, pulmonary, or metabolic disease); Major surgical procedure or significant traumatic injury within 28 days before enrollment or anticipation of the need for major surgery during the course of study treatment; Current pregnancy or lactation; Current known infection with HIV, active hepatitis B, and/or hepatitis C virus.

Efficacy Analyses

No adjustments for multiplicity of endpoints or within-subgroup comparisons will be incorporated in the efficacy analyses. The primary analysis population will be based on the treated population, which is defined as patients who received at least one dose of study drug. In addition, as a sensitivity analysis, the primary endpoint will be assessed in the efficacy-evaluable population, which is defined as patients who receive at least one dose of study drug and undergo at least one post-baseline response assessment, which includes, at a minimum, an assessment of all target lesions, or who die while on therapy. The secondary and exploratory efficacy analyses will be performed on the efficacy-evaluable population. The primary efficacy endpoint of this study is objective response, as assessed by independent radiologic review using RECIST. Objective response is defined as a complete or partial response determined on two consecutive occasions 4 weeks apart. An estimate of the objective response rate and 95% confidence intervals (Blyth-Still-Casella) will be calculated. The primary analysis population will be based on the treated population; for this analysis, patients without at least one post-baseline response assessment will be considered non-responders. In addition, objective response rate will be assessed in the efficacy-evaluable population, which is defined as patients who receive at least one dose of study drug and undergo at least one post-baseline response assessment, which includes, at a minimum, an assessment of all target lesions, or who die while on therapy.

Duration of objective response was assessed for patients with an objective response. Duration of objective response is defined as the time from the initial documentation of response to documented disease progression (including isolated CNS progression) or death from any cause on study. Separate analyses of duration of objective response will be performed based on IRF and investigator assessments. Methods for handling censoring for analysis are the same as described below for PFS.

Progression-Free Survival: PFS is defined as the time from the first day of treatment to documented disease progression (including isolated CNS progression) or death from any cause on study, whichever occurs first. Death on study is defined as death from any cause within 30 days of the last dose of T-DM1. Separate analyses of PFS will be performed based on IRF and investigator assessments. PFS will be estimated for efficacy-evaluable patients only. PFS data for patients without disease progression or death will be censored at the time of the last tumor assessment. Kaplan-Meier estimates of median PFS and PFS rates at 6 and 9 months will be reported as appropriate.

Clinical Benefit Rate (CBR) is defined as the proportion of patients with a complete or partial response or stable disease at 6 months. Patients without at least one post-baseline response assessment will be considered as experiencing no clinical benefit. CBR will be calculated separately for tumor assessments based on investigator and an IRF assessment.

Safety Analyses: All patients who receive any amount of T-DM1 therapy will be included in the safety analyses. Safety will be assessed through summaries of adverse events, deaths, and changes in laboratory test results. All adverse events for all patients will be collected for the safety dataset. Verbatim descriptions of adverse events will be mapped to thesaurus terms. All recorded adverse event data will be listed by study site, patient, and cycle. All adverse events occurring on or after the first treatment will be summarized by mapped term, appropriate thesaurus levels, and NCI CTCAE, v3.0 toxicity grade. All serious adverse events will be listed separately and summarized. In addition, the incidence of symptomatic CHF and/or LVEF <40% will be summarized. Deaths reported during the study treatment period and those reported during follow-up after patient treatment discontinuation will be summarized. Laboratory data will be summarized by grade using the NCI CTCAE, v3.0 toxicity grade. Changes in LVEF over time will be summarized and listed by scheduled measurement time. The occurrence of antibodies to T-DM1 will be listed.

Pharmacokinetic and Pharmacodynamic Analyses: For T-DM1 and total trastuzumab (T-DM1 and unconjugated trastuzumab), descriptive statistics, including mean and median trough and peak values, will be summarized. The following PK parameters will be estimated following the first through fourth doses and every other dose thereafter: AUC, maximum serum concentration, CL, volume of distribution, and half-life. For unconjugated DM1, levels will be summarized for each patient at each timepoint and the Cₘₐₓ estimated. For all three analytes, concentrations below the lower limit of quantification of the assay will be excluded or assigned a numeric value based on the lower reporting limit of the assay. Serum concentrations of T-DM1 and total trastuzumab (T-DM1 and unconjugated trastuzumab) and plasma levels of DM1 will be listed by patient and summarized descriptively (mean, standard deviation, percent coefficient of variation, minimum, and maximum). Individual and mean concentration versus time plots will be presented on both linear and logarithmic scales. PK parameters will be determined using the best available techniques that can be applied to all available data. Non-compartmental, compartmental-based, and population analysis methods will be considered.

Exploratory diagnostic analyses include the following: HER2 gene amplification levels in archival tumor tissue; HER2 mRNA expression levels in archival tumor tissue; Levels of expression of other HER family receptors and ligands in archival tumor tissue; Fcγ receptor and/or β1 tubulin polymorphisms; Troponin I as a prognostic marker of cardiac dysfunction. The utility of the HER family receptors and ligands and Fcγ receptors to predict response to T-DM1 or to act as biomarkers of T-DM1 activity following therapy will be assessed. Exploratory analyses will further refine investigation of the relationship between clinical outcome (e.g., objective response and PFS) and pre-treatment HER2 expression levels. The diagnostic markers will be assessed both on the continuous and discrete (subdivided into categories other than quartiles) scale for this purpose. The utility of the two polymorphisms to predict the incidence and severity of thrombocytopenia and polymorphism status will be assessed.

Patient-Reported Outcome Assessments: The FACT-B, as well as the FACT-B subscale (TOI-PFB), and Patient's Assessment of Pain will be used to explore the impact of T-DM1 on patient-reported symptoms. Mean scores and change from baseline to each timepoint will be assessed for all efficacy-evaluable patients, responders, and patients with stable disease or who are non-responders. The proportion of patients who have a clinically significant change in TOI-PFB scores at each timepoint will also be assessed. A change of 5 points in the TOI-PFB score is considered clinically significant.

Differences in Symptoms in Clinical Responders and Non-Responders: As an exploratory endpoint, differences in symptom progression between clinical responders and non-responders will be compared. Patients for whom no baseline TOI-PFB score is available, or for whom no post-baseline TOI-PFB score is available, will be excluded from this analysis.

Safety Profile of T-DM1 in Patients Who Develop Isolated and Treatable Brain Metastases. An exploratory assessment of the safety of T-DM1 was given to patients with isolated and treatable CNS progression. Adverse events, serious adverse events, and deaths in this patient subgroup will be summarized.

Missing Data: For objective response, patients without a post-baseline tumor assessment will be considered non-responders. For duration of response and PFS, data from patients who are lost to follow-up will be included in the analysis as censored observations on the last date that the patient was known to be progression-free, defined as the date of the last tumor assessment.

Determination of Sample Size: This study was designed to determine the efficacy and safety of T-DM1 in patients with HER2-positive MBC who have progressed while receiving HER2-directed therapy. The emphasis of the efficacy analysis will be on the estimation of the magnitude of the response rate and duration of response. At least 100 patients were enrolled in this Phase II study. In patients with HER2-positive MBC who have progressed while receiving HER2-directed therapy, a response rate of 25% to single-agent T-DM1 would be of interest; however, a response rate of 14% would be considered to represent equivocal clinical benefit. The sample size of 100 patients, calculated using the exact method for a single proportion, was chosen to ensure that there is at least 80% power to reject a null hypothesis of a response rate of 14% against an alternative of 25%. Given this sample size, the 95% confidence interval of an observed response rate of 25% would be 16.5%-33.5%, thus excluding 14% as the lower limit. This trial will not have adequate power to detect statistically meaningful differences in response rates by biomarker (PK/PD biomarkers or HER2 ligands and receptors) classification. The sample size was estimated using nQuery software.

The clinical study concluded with 110 patients receiving at least one dose of T-DM1, 91 of which had central HER2 data available. Of those, 76 (83.5%) were confirmed HER2+ retrospectively. Further baseline characteristics of the patient population include those in Table 1.

TABLE 1

| | Initial Analysis | |
|---|---|---|
| Characteristics | (Updated Analysis) | |
| Median age, years (range) | 52.5 | (34-77) |
| Median time since metastatic diagnosis, months | 41.4 | (1-149) |
| (range) | 42.1 | (1-149) |
| ECOG PS, n (%) | | |
| 0 | 54 | (49.1%) |
| 1 | 53 | (48.2%) |
| 2 | 3 | (2.7) |
| ER+ or PR+, n (%) | 55 | (50.0%) |
| ER− and PR−, n (%) | 51 | (46.4%) |
| Number of distinct metastatic sites, n (%) | | |
| <3 | 29 | (26.4%) |
| ≧3 | 81 | (73.6%) |
| Site of metastasis (≧40% patients) n (%) | | |
| Local-regional | 70 | (63.6%) |
| Lung | 69 | (62.7%) |
| Bone | 57 | (51.8%) |
| Liver | 49 | (44.5%) |

Median follow-up is 8.3 months (range 0.7-13.1). As of the data cutoff date, 40 patients were on study. Of the 70 patients that discontinued treatment: 58 had progressive disease (52.7%) and 6 had one or more adverse events (5.5%).

Also, 109 of the 110 patients had received prior trastuzumab, capecitabine, anthracycline, taxane and lapatinib. The prior chemotherapy and anti-HER2 therapy data of the patient population includes those in Table 2.

TABLE 2

| | Initial Analysis | |
|---|---|---|
| Prior therapy | (Updated Analysis) | |
| Median number of agents for metastatic | 7.0 | (1-15) |
| disease (range)* | 7.0 | (3-17) |
| Median number of agents in all therapy | 8.0 | (1-19) |
| setting (range)* | 8.5 | (5-19) |
| Number of patients with 5 prior agents | 109 | (99.1) |
| (trastuzumab, capecitabine, anthracycline, | | |
| taxane and lapatinib), n(%) | | |
| Prior trastuzumab | | |
| Median duration of prior trastuzumab in | 19.4 | (2-116) |
| metastatic setting, months (range) | 19.7 | (1.8-115.8) |
| Prior lapatinib | | |
| Median duration of prior lapatinib in | 6.9 | (0-23) |
| metastatic setting, months (range) | 6.8 | (0.2-23.3) |

*Includes all agents intended for the treatment of breast cancer except hormonal therapy

The pharmacokinetics (PK) parameters for T-DM1 conjugate in this phase II study were similar to those reported at the MTD in the phase I study, and to PK parameters for the previously reported 112 patient proof-of-concept Phase II Study. maximum serum T-DM1 concentration (Cₘₐₓ, μg/ml=76.8, terminal halflife in days (t½)=4.0, Clearance (CL, ml/day/kg)=8.3.

Patient exposure to drug data for the 110 patients included those in Table 3.

TABLE 3

| | Initial Analysis | |
|---|---|---|
| Exposure to Drug | (Updated Analysis) | |
| # Doses Administered, Median (Range) | 7.0 | (1-19) |
| | 7.0 | (1-30) |
| Exposure Duration (weeks), Median (Range) | 19.3 | (0-56) |
| | 19.3 | (0.1-88) |
| Average T-DM1 Dose (mg/kg), Median (Range) | 3.57 | (2.5-3.9) |
| | 3.56 | (2.5-3.9) |

| | |
|---|---|
| Number of Patients with Dose Reductions to | 11 |
| 3.0 mg/kg* | |
| Number of Patients with Dose Reductions to | 6 |
| 2.4 mg/kg* | 7 |

*Reasons for Dose Reduction Include: Grade 4 Thrombocytopenia (1), Grade 3 Elevation AST + ALT (2), Grade 3 AST Elevation (1), Grade 2 Thrombocytopenia (1), Grade 2 Leukopenia (2), Grade 2 AST + ALT (3), Grade 2 AST (1), Grade 1 AST (1).

### Example 5

### Assessments During Treatment

Screening clinical evaluations and laboratory assessments may be used as the Cycle 1, Day 1 evaluations if performed within 96 hours preceding T-DM1 administration. Unless specified otherwise, all procedures and assessments should be performed before the T-DM1 infusion. Local laboratory assessments scheduled for Day 1 of Cycles 2 and beyond may be performed within 72 hours preceding T-DM1 administration unless specified otherwise. Results of local laboratory assessments must be reviewed and the review documented before T-DM1 administration.

Cycle 1, Day 1 (±3 days):

A limited physical examination is intended to be part of an interim safety evaluation, and should focus on organ systems that are related to a potential AE as suggested by a patient's interim medical history and/or existing clinical and preclinical data for T-DM1 to measure: Weight; Vital signs (blood pressure, heart rate, and temperature): pre-dose, every 15 (±5) minutes during the first T-DM1 infusion, and 90 (±10) minutes after the end of infusion); ECOG performance status; Concomitant medications; and Adverse events.

Local laboratory assessments (pre-dose) included: Complete blood count (CBC) with platelet count and differential; Serum chemistry (BUN, creatinine, total bilirubin, alkaline phosphatase, LDH, AST, and ALT). Central laboratory assessments included: Troponin I; Serum sample for measurement of HER2 ECD: pre-dose; Serum sample for anti-T-DM1 antibody analysis: pre-dose; Serum and plasma PK samples: pre-dose and post-dose at 30 (±10) minutes after the end of the T-DM1 infusion; Whole blood sample for FcγR and β1 tubulin genetic polymorphism (pre-dose). The blood sample was sent to the genotyping laboratory, where DNA will be isolated from whole blood and stored until the time of analysis. This sample will be used for analysis of FcγR and β1 tubulin polymorphisms only.

T-DM1 infusion: The first infusion will be administered over 90 (±10) minutes. Patients will be monitored for any untoward effects for 90 minutes following completion of the T-DM1 infusion (see Section 4.3.2).

Cycle 1, Day 8 (±1 day):

Local laboratory assessments included: CBC with platelet count and differential. Patients with Grade 3 or 4 thrombocytopenia should have additional CBC(s) within 5 days to determine the duration of thrombocytopenia; Serum chemistry (BUN, creatinine, total bilirubin, alkaline phosphatase, LDH, AST, and ALT). Central laboratory assessments included: Serum and plasma PK samples; Troponin I

Cycle 1, Day 15 (±1 day):

Local laboratory assessment included: CBC with platelet count and differential. Patients with Grade 3 or 4 thrombocytopenia should have additional CBC(s) within 5 days to determine the duration of thrombocytopenia. Central laboratory assessments included: Serum and plasma PK samples

Cycles 2-17, Day 1 (±3 days):

FACT-B and Patient's Assessment of Pain administration (only in Cycles 2, 4, 6, 8, 10, 12, 14, and 16; before any other Day 1 procedures). Administer before any other procedures or discussion of tumor assessment results, included Limited physical examination; Serum albumin (Cycle 4 only); Weight; Vital signs (blood pressure, heart rate, and temperature): pre-dose and 30 (±10) minutes after the end of infusion); ECOG performance status; Concomitant medications; Adverse events. Local laboratory assessments (pre-dose) included: CBC with platelet count and differential; Serum chemistry (BUN, creatinine, total bilirubin, alkaline phosphatase, LDH, AST, and ALT). Central laboratory assessments included: Serum samples for anti-T-DM1 antibody analysis: pre-dose (Cycles 2, 3, and 4); Serum and plasma PK samples: pre-dose and post-dose at 30 (±10) minutes after end of the T-DM1 infusion (Cycles 2, 3, and 4; and at Cycles 6, 8, 12, and 16). If prior infusions of T-DM1 occurred without signs or symptoms of infusion reactions, subsequent infusions may be administered over ˜30 (±10) minutes, and patients will be monitored for any untoward effects for 30 minutes following completion of the T-DM1 infusion.

Cycles 2-17, Day 8 (±1 day):

Local laboratory assessments included CBC with platelet count and differential; Patients with Grade 3 or 4 thrombocytopenia should have additional CBC(s) within 5 days to determine the duration of thrombocytopenia; Serum chemistry (BUN, creatinine, total bilirubin, alkaline phosphatase, LDH, AST, and ALT). Central laboratory assessments included: Serum and plasma PK samples (Cycle 4); Troponin I

Cycles 2-17, Day 15 (±1 day):

Central laboratory assessments included: Serum and plasma PK samples (Cycle 4)

Weeks 6, 12, 18, 24, 30, 36, 42, and 48 (±5 days) until Disease Progression or Study Completion: Tumor assessments were be performed every 6 weeks from the first day of dosing until disease progression regardless of drug delays or interruptions or early drug discontinuation. Objective responses must be confirmed at least 28 days after the initial documentation of response. Tumor burden must be evaluated by physical examination and image-based evaluation (per RECIST). Assessments should include an evaluation of all sites of disease. A CT or MRI scan of the chest, abdomen, and pelvis is required. Contrast agents (IV and oral) should be used per standard practice. Bone scans and brain MRI or CT scans should be performed when clinically indicated and at the discretion of the investigator. The same radiographic procedure used to define measurable disease sites at baseline must be used throughout the study (e.g., the same contrast protocol for CT scans). Technical imaging parameters will be defined in a separate Radiology Technical Manual that will be distributed to sites. An isotope bone scan or other radiographic imaging modality should be repeated or performed in the event of clinical suspicion of progression of existing bone lesions and/or the development of new bone lesions. All radiographs including bone scans obtained as part of the protocol-required tumor assessments will be submitted to an IRF, including interval assessments obtained that documented progression of disease if applicable or for confirmation of objective response, if performed. ECHO or MUGA scan (ECHO is preferred). The same method used at screening should be used throughout the study. Echocardiograms (or MUGA scans) will be submitted to the IRF.

Follow-Up or Early Termination Visit: Upon completion of treatment, patients must return for a follow-up visit within 30 days after the last T-DM1 dose. Patients who discontinue before completing treatment are required to return to the clinic for an early termination visit within 30 days after the last T-DM1 dose. The visit at which a tumor assessment shows disease progression may be used as the early termination visit. The following assessments should be performed at the follow-up visit or early termination visit: FACT-B (for female patients only) and Patient's Assessment of Pain administered before any procedures are conducted and results are discussed with the patient; Limited physical examination; Weight; Vital signs (blood pressure, heart rate, and temperature); ECOG performance status; Concomitant medications; Adverse events; 12-lead ECG; Echocardiogram or MUGA scan (if the most recent follow-up scan was performed 30 days before termination of participation, or if no post-treatment scan has been performed); Echocardiograms (or MUGA scans) will be submitted to the IRF.

Tumor assessment, if PD not previously documented on imaging studies: Tumor burden must be evaluated through image-based evaluation (per RECIST). Use the same radiologic procedures to define measurable disease sites as those used at baseline (e.g., the same contrast for CT scans). Radiographs will be submitted to the IRF.

Local laboratory assessments included: CBC with platelet count with differential; Serum chemistry (glucose, BUN, creatinine, sodium, potassium, chloride, bicarbonate, calcium, uric acid, total protein, albumin, total bilirubin, alkaline phosphatase, LDH, AST, and ALT); INR and aPTT; Laboratory urinalysis. Central laboratory assessments included: Serum sample for anti-T-DM1 antibody analysis, Serum and plasma PK samples; Troponin I.

Additional Safety Follow-Up: Patients will be followed for adverse events for 30 days following the last infusion of T-DM1 or until the early termination visit, or initiation of another anti-cancer therapy, whichever occurs first. In addition, patients will be contacted regarding the occurrence of all serious adverse events 60 and 90 days following the last T-DM1 infusion or until initiation of another anti-cancer therapy, whichever occurs first.

### Example 6

### Assay Methods

Serum HER2 Extracellular Domain: The Oncogene Science (Bayer) HER2 ELISA is a sandwich enzyme immunoassay that utilizes a mouse monoclonal antibody for capture and a different biotinylated mouse monoclonal antibody for the detection of human HER2 protein. Both capture and detector reagents specifically bind to the ECD of HER2 protein. The capture antibody has been immobilized on the interior surface of microtiter plate wells. To perform the test, an appropriate volume of specimen is incubated in the coated well to allow binding of the antigen by the capture antibody. The immobilized antigen is then reacted with the detector antiserum. The amount of detector antibody bound to antigen is measured by binding it with a streptavidin/horseradish peroxidase conjugate, which then catalyzes the conversion of the chromogenic Substrate o-phenylenediamine into a colored product. The colored reaction product is quantitated by spectrophotometry and is related to the amount of HER-2/neu protein in the sample.

HER2 Testing: HER2 gene amplification will be assessed on archival tumor material using the standard Vysis PathVysion® HER2 FISH kit according to the instructions in the HERCEPTIN® Package Insert. Briefly, 5-micron tissue sections will be heated in formamide to denature the DNA. Dual-color DNA probes for HER2 (red) and CEP17 (green) will be applied to the slide for hybridization. The slides will then be washed to remove unbound probe. The average number of HER2 and CEP17 copies per cell will be determined by counting signals in ≧20 tumor nuclei. The ratio of HER2 to CEP17 is then calculated. If the ratio is ≧2.0, the sample is considered amplified. In some cases, tumor samples may also be tested for HER2 protein overexpression by IHC using the DAKO HercepTest™ kit according to the instructions in the HERCEPTIN® Package Insert.

T-DM1 ELISA: PK sampling will be performed at the timepoints shown in the study flowchart; total trastuzumab and T-DM1 samples were assayed. Serum samples will be assayed for T-DM1 in an indirect sandwich ELISA. The assay will use an anti-DM1 monoclonal antibody in the capture phase of the assay. The assay will use biotinylated recombinant HER2 ECD and horseradish peroxidase conjugated to streptavidin for detection. The minimum quantifiable concentration is 40 ng/mL.

Total Trastuzumab ELISA: Serum samples will be assayed for total trastuzumab (trastuzumab and trastuzumab conjugated to DM1) in an indirect sandwich ELISA. The assay will use recombinant HER2 ECD in the capture phase of the assay and horseradish peroxidase conjugated to F(ab′)2 goat anti-human IgG Fcγ for detection. The minimum quantifiable concentration is 40 ng/mL.

Free DM1 LC-MS/MS Assay: DM1 samples were assayed by mass spectrometry (Xendo Groningen, the Netherlands). Lithium-heparin plasma samples will be assayed using electrospray liquid chromatography tandem mass spectrometry (LC-MS/MS). Before assaying, the samples will be treated with a reducing agent to release disulfide-bound DM1 followed by treatment with n-ethyl maleimide to block the free sulfhydryls. The free DM1 will then be extracted from the plasma and assayed by LC-MS/MS. The lower and upper limits of quantification are 1.00 nmol/L and 500 nmol/L, respectively.

Anti-T-DM1 Antibody Electrochemiluminescence Assay: Serum samples will be assayed for anti-T-DM1 antibodies in a bridging antibody electrochemiluminescence assay. Samples, biotinylated T-DM1, and T-DM1 conjugated to an electrochemiluminescent tag will be incubated together to form the antibody bridge. Streptavidin-coated paramagnetic beads will be used to capture the complex for detection by electrochemiluminescence. The minimum dilution of serum samples is 1/10, and the sensitivity of the assay is log₁₀ 10 (equivalent to 1.0-log titer unit).

Troponin I: Serum troponin I will be sent to a central laboratory for an exploratory analysis to investigate the role of cardiac troponin I as a prognostic marker for heart failure for patients treated with T-DM1.

Whole Blood and Tumor Tissue Samples: Whole blood samples were obtained from patients at the timepoints shown and samples were sent to a central laboratory for assay.

Paraffin block tumor tissue and/or slides from patients will be collected at the timepoints shown in the study flowchart and sent to a contract research organization for HER2 FISH (Persons D L, Bui M M, Lowery M C, et al. Fluorescence in situ hybridization (FISH) for detection of HER-2/neu amplification in breast cancer: a multicenter portability study. (2000) Ann Clin Lab Sci 30:41-8), IHC, and quantitative RT-PCR analysis, as well as quantitative RT-PCR analysis of other HER family receptors and/or ligands. Some samples may be enriched for tumor content by macrodissection of histologically identifiable tumor. RNA will be extracted, and quantitative RT-PCR for HER family receptors and/or ligands and a reference gene will be performed using a standard platform (e.g., LightCycler or TaqMan®).

Safety Outcome Measures

The safety and tolerability of T-DM1 was assessed using the following primary safety outcome measures: (1) Incidence, nature, and severity of adverse events; (2) Adverse events or changes in physical findings and clinical laboratory results during and following study drug administration that result in dose modification, dose delay, or discontinuation of T-DM1 and/or pertuzumab; and (3) Change in cardiac function (i.e., left ventricular ejection fraction [LVEF], segmental wall abnormalities), including ECHO or MUGA scans

Pharmacokinetic and Pharmacodynamic Outcome Measures

The following pharmacokinetic parameters of T-DM1 were determined in all patients who receive study treatment using either non-compartmental and/or population methods, when appropriate, as data allowed: (1) Serum concentrations of T-DM1 (conjugate), total trastuzumab (free and conjugated to DM1); (2) Plasma concentrations of free DM1; (3) Total exposure (area under the concentration-time curve [AUC]); (4) Maximum serum concentration (Cmax); (5) Minimum concentration (Cmin); (6) Clearance; (7) Volume of distribution; (8) Terminal half-life; (9) Anti-therapeutic antibodies to T-DM1.

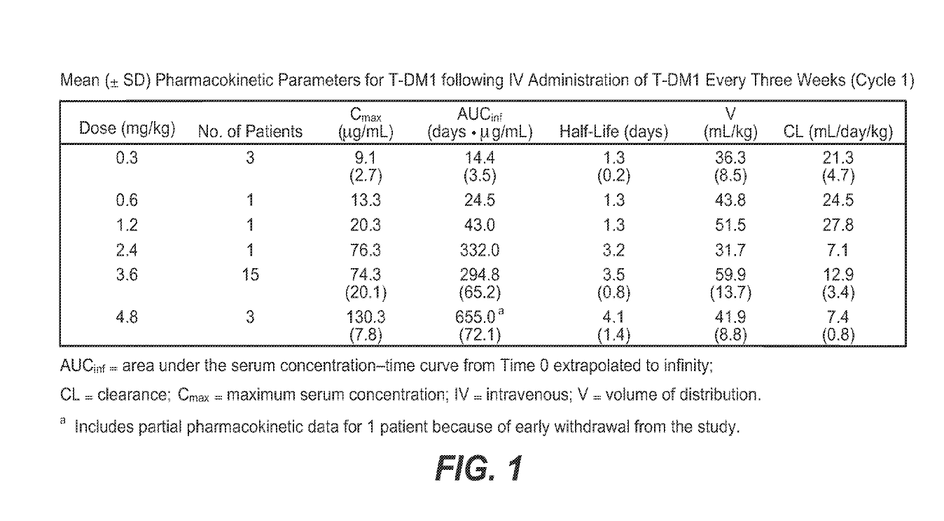

FIG. 1 shows Mean (±SD) Pharmacokinetic Parameters for T-DM1 following IV Administration of T-DM1 Every Three Weeks (Cycle 1).

Efficacy Outcome Measures

The objective response rate using modified RECIST, v1.0 was assessed as the efficacy outcome measure. The secondary efficacy outcome measures of this study are the following: (1) PFS, defined as the time from the study treatment initiation to the first occurrence of disease progression or death on study (within 30 days of the last dose of study treatment) from any cause, as determined by investigator review of tumor assessments using modified RECIST, v1.0; and (2) Duration of response, defined as the first occurrence of a documented objective response until the time of disease progression, as determined by investigator review of tumor assessments using modified RECIST (v1.0), or death on study (within 30 days of the last dose of study treatment) from any cause.

Statistical Methods

Continuous data was summarized using mean, standard deviation, median, minimum, and maximum. Discrete data will be summarized using frequencies and percentages.

### Example 7

### Response Evaluation Criteria in Solid Tumors (RECIST)

The Response Evaluation Criteria in Solid Tumors (RECIST) was applied according to: Therasse P, Arbuck S G, Eisenhauser E A, Wanders J, Kaplan R S, Rubinstein L, et al. New guidelines to evaluate the response to treatment in solid tumors. J Natl Cancer Inst 2000; 92:205-16); and as follows.

Measurability Of Tumor Lesions At Baseline: At baseline, tumor lesions will be categorized as follows: measurable (lesions that can be accurately measured in at least one dimension [longest diameter to be recorded] as 20 mm with conventional techniques or as 10 mm with spiral CT scan [see section 2.2]) or nonmeasurable (all other lesions, including small lesions [longest diameter <20 mm with conventional techniques or <10 mm with spiral CT scan] and truly nonmeasurable lesions). The term “evaluable” in reference to measurability is not recommended and will not be used because it does not provide additional meaning or accuracy. All measurements should be recorded in metric notation by use of a ruler or calipers. All baseline evaluations should be performed as closely as possible to the beginning of treatment and never more than 4 weeks before the beginning of treatment. Lesions considered to be truly nonmeasurable include the following: bone lesions, leptomeningeal disease, ascites, pleural/pericardial effusion, inflammatory breast disease, lymphangitis cutis/pulmonis, abdominal masses that are not confirmed and followed by imaging techniques, and cystic lesions. (Note: Tumor lesions that are situated in a previously irradiated area might or might not be considered measurable, and the conditions under which such lesions should be considered must be defined in the protocol when appropriate.

Specifications by Methods of Measurements: The same method of assessment and the same technique should be used to characterize each identified and reported lesion at baseline and during follow-up. Imaging-based evaluation is preferred to evaluation by clinical examination when both methods have been used to assess the antitumor effect of a treatment.

Clinical Examination: Clinically detected lesions will only be considered measurable when they are superficial (e.g., skin nodules and palpable lymph nodes). For the case of skin lesions, documentation by color photography—including a ruler to estimate the size of the lesion—is recommended.

Chest X-ray: Lesions on chest X-rays are acceptable as measurable lesions when they are clearly defined and surrounded by aerated lung. However, CT is preferable. More details concerning the use of this method of assessment for objective tumor response evaluation are provided in Therasse P, Arbuck S G, Eisenhauser E A, Wanders J, Kaplan R S, Rubinstein L, et al. New guidelines to evaluate the response to treatment in solid tumors. (2000) J Natl Cancer Inst 92:205-16.

CT and MRI: CT and MRI are the best currently available and most reproducible methods for measuring target lesions selected for response assessment. Conventional CT and MRI should be performed with contiguous cuts of 10 mm or less in slice thickness. Spiral CT should be performed by use of a 5-mm contiguous reconstruction algorithm; this specification applies to the tumors of the chest, abdomen, and pelvis, while head and neck tumors and those of the extremities usually require specific protocols.

Ultrasound: When the primary endpoint of the study is objective response evaluation, ultrasound should not be used to measure tumor lesions that are clinically not easily accessible. It may be used as a possible alternative to clinical measurements for superficial palpable lymph nodes, subcutaneous lesions, and thyroid nodules. Ultrasound might also be useful to confirm the complete disappearance of superficial lesions usually assessed by clinical examination.

Endoscopy and Laparoscopy: The utilization of these techniques for objective tumor evaluation has not yet been fully or widely validated. Their uses in this specific context require sophisticated equipment and a high level of expertise that may be available only in some centers. Therefore, utilization of such techniques for objective tumor response should be restricted to validation purposes in specialized centers. However, such techniques can be useful in confirming complete histopathologic response when biopsy specimens are obtained.

Tumor Markers: Tumor markers alone cannot be used to assess response. However, if markers are initially above the upper normal limit, they must return to normal levels for a patient to be considered in complete clinical response when all tumor lesions have disappeared. Specific additional criteria for standardized usage of prostate-specific antigen and CA (cancer antigen) 125 response in support of clinical trials are being validated.

Cytology and Histology: Cytologic and histologic techniques can be used to differentiate between partial response and complete response in rare cases (e.g., after treatment to differentiate between residual benign lesions and residual malignant lesions in tumor types such as germ cell tumors). Cytologic confirmation of the neoplastic nature of any effusion that appears or worsens during treatment is required when the measurable tumor has met criteria for response or stable disease. Under such circumstances, the cytologic examination of the fluid collected will permit differentiation between response or stable disease (an effusion may be a side effect of the treatment) and progressive disease (if the neoplastic origin of the fluid is confirmed). New techniques to better establish objective tumor response will be integrated into these criteria, when they are fully validated, to be used in the context of tumor response evaluation.

Tumor Response Evaluation and Assessment of Overall Tumor Burden and Measurable Disease: To assess objective response, it is necessary to estimate the overall tumor burden at baseline to which subsequent measurements will be compared. Only patients with measurable disease at baseline should be included in protocols where objective tumor response is the primary endpoint. Measurable disease is defined by the presence of at least one measurable lesion (as defined in Section 2.1). If the measurable disease is restricted to a solitary lesion, its neoplastic nature should be confirmed by cytology/histology.

Baseline Documentation of “Target” and “Nontarget” Lesions: All measurable lesions up to a maximum of 5 lesions per organ and 10 lesions in total, representative of all involved organs, should be identified as target lesions and recorded and measured at baseline. Target lesions should be selected on the basis of their size (those with the longest diameter) and their suitability for accurate repeated measurements (either by imaging techniques or clinically). A sum of the longest diameter for all target lesions will be calculated and reported as the baseline sum longest diameter. The baseline sum longest diameter will be used as the reference by which to characterize the objective tumor response. All other lesions (or sites of disease) should be identified as nontarget lesions and should also be recorded at baseline. Measurements of these lesions are not required, but the presence or absence of each should be noted throughout follow-up.

Response Criteria and Evaluation of Target Lesions: Criteria have been adapted from the original WHO Handbook, taking into account the measurement of the longest diameter only for all target lesions: complete response—the disappearance of all target lesions; partial response—at least a 30% decrease in the sum of the longest diameter of target lesions, taking as reference the baseline sum longest diameter; progressive disease—at least a 20% increase in the sum of the longest diameter of target lesions, taking as reference the smallest sum longest diameter recorded since the treatment started or the appearance of one or more new lesions; stable disease—neither sufficient shrinkage to qualify for partial response nor sufficient increase to qualify for progressive disease, taking as reference the smallest sum longest diameter since the treatment started.

Evaluation of Nontarget Lesions: The definitions of the criteria used to determine the objective tumor response for nontarget lesions include: complete response—the disappearance of all nontarget lesions and normalization of tumor marker level; incomplete response/stable disease—the persistence of one or more nontarget lesion(s) and/or the maintenance of tumor marker level above the normal limits; and progressive disease—the appearance of one or more new lesions and/or unequivocal progression of existing nontarget lesions. (Note: Although a clear progression of “nontarget” lesions only is exceptional, in such circumstances, the opinion of the treating physician should prevail and the progression status should be confirmed later by the review panel [or study chair].

Primary analysis of the antitumor activity data included those in Tables 4A and 4B:

TABLE 4A
reflecting initial analysis

| | | |
|---|---|---|
| | IRF | Investigator |
| Tumor Response | (N = 110) | (N = 110) |

| | | | | |
|---|---|---|---|---|
| Objective Response | 36 | (32.7%) | 33 | (30.0%) |
| Rate, n (%) | | | | |

| (95% CI) | (24.1-42.1) | (22.0-39.4) | |
|---|---|---|---|
| CR | 0 | 2 | (1.8%) |

| | | | | |
|---|---|---|---|---|
| PR | 36 | (32.7%) | 31 | (28.2%) |
| SD* | 51 | (46.4%) | 58 | (52.7%) |
| PD | 20 | (18.2%) | 15 | (13.6%) |
| UE | 2 | (1.8%) | 1 | (0.9%) |
| Missing | 1 | (0.9%) | 3 | (2.7%) |
| Clinical Benefit | 49 | (44.5%) | 44 | (40%) |
| Rate, n (%) | | | | |

| | | |
|---|---|---|
| (95% CI) | (35.1-54.3) | (31.1-49.3) |

IRF—Independent Review Facility
Objective Response—CR or PR determined by two consecutive tumor assessments at least 28 days apart
Clinical Benefit—objective response or SD maintained for at least 6 months.
*Including unconfirmed PRs

TABLE 4B
reflecting updated analysis

| | IRF | Investigator |
|---|---|---|
| Tumor Response | (N = 110) | (N = 110) |
| Objective Response | 34.5% | 32.7% |
| Rate, % | | |
| (95% CI) | (26.1-43.9) | (24.1-42.1) |
| CR | 0 | 4.5% |
| PR | 34.5% | 28.2% |
| SD* | 44.5% | 50.9% |
| PD | 18.2% | 14.5% |
| UE | 1.8% | 0.9% |
| Missing | 0.9% | 0.9% |
| Clinical Benefit | 48.2% | 46.4% |
| Rate, n (%) | | |
| (95% CI) | (38.8-57.9) | (37.1-56.1) |

IRF—Independent Review Facility
Objective Response—CR or PR determined by two consecutive tumor assessments at least 28 days apart
Clinical Benefit—objective response or SD maintained for at least 6 months.
*Including unconfirmed PRs

The antitumor activity in treated HER2 normal and HER2 positive patients by retrospectively confirmed HER2 status includes that in Table 5:

TABLE 5
reflecting initial analysis, except as indicated

| | IRF | | INV | |
|---|---|---|---|---|
| | (updated analysis) | | (updated analysis) | |
| Patients with HER2+ | n = 76 | (n = 80) | n = 76 | (n = 80) |
| ORR, % | 39.5 | (41.3%) | 36.8 | (40.0%) |
| Clinical benefit rate, % | 52.6 | (55.0%) | 47.4 | (53.8%) |

| | | | |
|---|---|---|---|
| Patients with HER2 normal | n = 15 | n = 15 | (n = 14) |

| | | |
|---|---|---|
| ORR, % | 20.0 | 13.3 |
| Clinical benefit rate, % | 26.7 | 20.0 |

IRF—Independent Review Facility; INV—Investigator; Objective Response—CR or PR determined by two consecutive tumor assessments at least 28 days apart; Clinical Benefit—objective response or SD maintained for at least 6 months

FIG. 3 shows a Kaplan-Meier plot of progression-free survival (PFS) in all treated patients. An updated analysis yielded similar results, with median PFS of 6.9 (IRF) and 95% CI (4.2-8.4). FIG. 4 shows a Kaplan-Meier plot of progression-free survival (PFS) in patients separated by HER2 status. FIG. 5 shows a Kaplan-Meier plot of PFS per IRF Assessment by HER2 qRT-PCR for Retrospectively Confirmed HER2+ treated patients.

A summary of IRF and investigator (INV) assessment includes those in Table 6:

TABLE 6

| Patients | IRF | INV |
|---|---|---|
| Progressed | 50 (45.5%) | 60 (54.5%) |
| Censored | 60 (54.5%) | 50 (45.5%) |
| PFS (months) median | 7.3 | 5.5 |
| 95% CI of median | 4.2 | 4.2, 7.9 |
| 25-75% | 2.6-9.7 | 2.8-11.1 |
| range | 0.0+-11.7+ | 0.0+-12.7+ |

Evaluation of Best Overall Response: The best overall response is the best response recorded from the start of treatment until disease progression/recurrence (taking as reference for progressive disease the smallest measurements recorded since the treatment started). In general, the patient's best response assignment will depend on the achievement of both measurement and confirmation criteria. Table 7 provides overall responses for all possible combinations of tumor responses in target and nontarget lesions with or without the appearance of new lesions.

TABLE 7

| Target | Nontarget | New | Overall |
|---|---|---|---|
| Lesions | Lesions | Lesions | Response |
| CR | CR | No | CR |
| CR | Incomplete | No | PR |
| | response/SD | | |
| PR | Non-PD | No | PR |
| SD | Non-PD | No | SD |
| PD | Any | Yes or No | PD |
| Any | PD | Yes or No | PD |
| Any | Any | Yes | PD |

* CR = complete response; PR = partial response; SD = stable disease; and PD = progressive disease.
a. Patients with a global deterioration of health status requiring discontinuation of treatment without objective evidence of disease progression at that time should be classified as having “symptomatic deterioration.” Every effort should be made to document the objective disease progression, even after discontinuation of treatment.
b. Conditions that may define early progression, early death, and inevaluability are study specific and should be clearly defined in each protocol (depending on treatment duration and treatment periodicity).
c. In some circumstances, it may be difficult to distinguish residual disease from normal tissue. When the evaluation of complete response depends on this determination, it is recommended that the residual lesion be investigated (fine-needle aspiration/biopsy) before confirming the complete response status.

Frequency of Tumor Re-Evaluation: Frequency of tumor re-evaluation while on treatment should be protocol specific and adapted to the type and schedule of treatment. However, in the context of Phase II studies where the beneficial effect of therapy is not known, follow-up of every other cycle (i.e., 6 to 8 weeks) seems a reasonable norm. Smaller or greater time intervals than these could be justified in specific regimens or circumstances. After the end of the treatment, the need for repetitive tumor evaluations depends on whether the Phase II trial has, as a goal, the response rate, or the time to an event (disease progression/death). If time to an event is the main endpoint of the study, then routine re-evaluation is warranted of those patients who went off the study for reasons other than the expected event at frequencies to be determined by the protocol. Intervals between evaluations twice as long as on study are often used, but no strict rule can be made.

Confirmatory Measurement/Duration of Response: The main goal of confirmation of objective response in clinical trials is to avoid overestimating the response rate observed, useful in nonrandomized trials where response is the primary endpoint. In this setting, to be assigned a status of partial response or complete response, changes in tumor measurements must be confirmed by repeat assessments that should be performed no less than 4 weeks after the criteria for response are first met. Longer intervals as determined by the study protocol may also be appropriate. In the case of stable disease, measurements must have met the stable disease criteria at least once after study entry at a minimum interval (in general, not less than 6 to 8 weeks) that is defined in the study protocol.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

We claim:

1. A method for the treatment of metastatic or unresectable locally advanced HER2 positive cancer in a patient comprising administering a therapeutically effective amount of trastuzumab-MCC-DM1, wherein the patient has been previously treated with at least two anti-HER2 agents.

2. The method of claim 1, wherein the at least two anti-HER2 agents are lapatinib and trastuzumab.

3. The method of claim 1 or 2, wherein the patient has been previously treated with an anthracycline, a taxane, and capecitabine.

4. The method of any of claims 1-3, wherein the cancer is breast cancer.

5. The method of claim 1, wherein trastuzumab-MCC-DM1 is administered at three week intervals to the patient.

6. The method of claim 1, wherein the therapeutically effective amount of trastuzumab-MCC-DM1 is 1 mg to 10 mg/kg/day of patient body weight.

7. The method of claim 1, wherein trastuzumab-MCC-DM1 is formulated with sodium succinate, pH 5.0, and 0.02% (w/v) polysorbate 20.

8. The method of claim 7, wherein trastuzumab-MCC-DM1 is further formulated with 6% (w/v) trehalose dihydrate or 6% (w/v) sucrose.

9. A method for the treatment of metastatic or unresectable locally advanced HER2 positive cancer in a patient comprising administering a therapeutically effective amount of trastuzumab-MCC-DM1, wherein the patient was previously treated with two or more therapies selected from an anthracycline, a taxane, capecitabine, lapatinib, and trastuzumab, and wherein the patient progressed on their most recent treatment.

10. The method of claim 9, wherein the cancer is breast cancer.

11. The method of claim 9, wherein trastuzumab-MCC-DM1 is administered at three week intervals to the patient.

12. The method of claim 9, wherein the therapeutically effective amount of trastuzumab-MCC-DM1 is 1 mg to 10 mg/kg/day of patient body weight.

13. The method of claim 9, wherein trastuzumab-MCC-DM1 is formulated with sodium succinate, pH 5.0, and 0.02% (w/v) polysorbate 20.

14. The method of claim 9, wherein trastuzumab-MCC-DM1 is further formulated with 6% (w/v) trehalose dihydrate or 6% (w/v) sucrose.
